(19) United States
(12) Patent Application Publication
(10) Pub. No.: US 2011/0105488 A1
(43) Pub. Date: May 5, 2011

(54) SUBSTITUTED PYRROLIDINE AND PIPERIDINE COMPOUNDS, DERIVATIVES THEREOF, AND METHODS FOR TREATING PAIN

(75) Inventors: Giorgio Attardo, Laval (CA); Sasmita Tripathy, Pierrefonds (CA)

(73) Assignee: Chlorion Pharma, Inc., Laval, QC (CA)

(21) Appl. No.: 12/933,719

(22) PCT Filed: Mar. 20, 2009
(86) PCT No.: PCT/CA09/00366

§ 371 Date: Dec. 20, 2010

Related U.S. Application Data
(60) Provisional application No. 61/038,662, filed on Mar. 21, 2008.

(51) Int. Cl. A61K 31/535; C07D 207/10; C07D 277/04; C07D 207/14; C07D 265/02; C07D 207/12; C07D 261/02; C07D 211/14; A61K 31/40; A61K 31/426; A61K 31/42; A61K 31/4453; A61P 29/02

(52) U.S. Cl. 514/228.8; 548/568; 548/146; 548/557; 544/63; 548/556; 548/240; 546/247; 548/187; 514/428; 514/365; 514/426; 514/424; 514/378; 514/315; 514/369

(57) ABSTRACT

The present application relates to pyrrolidine, piperidine, and other nitrogen-containing heterocyclic derivatives and the use of these compounds for treating and preventing pam or inflammation. The analgesic compounds demonstrate efficacy in the treatment of neuropathic pain resulting from a variety of conditions such as diabetic neuropathy, HIV infections, and post-herpetic neuralgia.

### CROSS-REFERENCE TO RELATED APPLICATIONS

This application claims benefit of U.S. Provisional Application No. 61/038,662, filed Mar. 21, 2008, which is hereby incorporated by reference.

### BACKGROUND OF THE INVENTION

In general, the present invention relates to substituted pyrrolidine and piperidine compounds, and derivatives thereof, and to the use of these compounds to treat pain.

Pain is a common form of physical suffering and distress and is one of the most common reasons patients report to physicians. It may be categorized in terms of form (nociceptive or neuropathic), duration (chronic or acute), and degree (mild, moderate or severe). Typically, nociceptive pain is acute and results from injury, such as burns, sprains, fractures, inflammation (inflammatory pain, including from osteo- and rheumatoid arthritis). Neuropathic pain, on the other hand, is defined by the International Association for the Study of Pain as a form of chronic pain that is caused by a lesion or dysfunction of the nervous system. Commonly, neuropathic pain results from diabetic neuropathy, HIV infections, and post-herpetic neuralgia. Other disorders that are associated with neuropathic pain include complex regional pain syndromes, trigeminal neuralgia, low back pain, sciatica, phantom limb pain, blast pain, and fibromyalgia.

Few therapeutics are approved by the US Food and Drug Administration and other regulatory agencies for the treatment of neuropathic pain. Therapeutics that are approved for this indication include gabapentin (post-herpetic neuralgia), pregabalin (post-herpetic neuralgia, diabetic peripheral neuropathy, fibromyalgia), carbamazepine (trigeminal neuralgia), duloxetine (diabetic peripheral neuropathy), and lidocaine patch (post-herpetic neuralgia). The first four therapeutics are administered systemically, whereas the last is administered locally (topically) to the site of hypersensitivity.

Generally, clinical investigations have shown that these in-market therapeutics evoke a maximum of 50% reduction in overall pain scores from baseline in patients with neuropathic pain resulting from a variety of conditions (DataMonitor, Pipeline Insight: Neuropathic Pain, 2005; Markham & Dworkin, Journal of Pain, 2006). For example, gabapentin, which is considered by many to be the “gold-standard” treatment for post-herpetic neuralgia, was shown in two placebo-controlled, parallel-group trials of 8-weeks duration to effect an approximate 33% decrease in pain (from a mean baseline of 6.3 to a mean endpoint of 4.2) (Bakonja et al. and Rowbotham et al., Journal of the American Medical Association, 1998; for review see Jensen, European Journal of Pain, 2002).

Moreover, the tolerability of in-market neuropathic pain therapeutics leaves much to be desired; in a recent review of multiple clinical studies, for example, pregabalin was reported to evoke significant somnolence, vertigo, and headache, and typically resulted in more than 3 in 10 subjects withdrawing from clinical trials (Tassone et al., Clinical Therapeutics, 2007). Similarly, carbamazepine is well recognized to evoke various sensory deficits, as well as rash and leucopenia in ˜10% of patients (Fuller et al., Expert Opinion on Drug Safety, 2006).

Taken together, it is clear that there is tremendous unmet medical need for new analgesic therapeutics with improved efficacy and tolerability in the treatment of the various forms of neuropathic pain.

### SUMMARY OF THE INVENTION

In general, the present invention provides compounds of Formula (Ia):

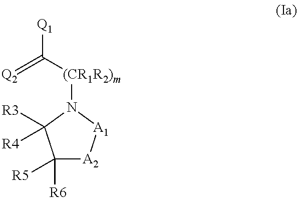

wherein:

A₁ is —O—, —S—, —SO—, —SO₂—, —CR₇R₈—, —NH—, —NR₉—, —NC(O)R₉—, —NSR₉—, —NSOR₉—, —NS(O)₂R₉—, —NC(S)R₉—, —NC(S)NHR₉—, —NC(S)NR₉R₁₀—, —NC(NH)NHR₉—, —NC(NH)NR₉R₁₀—, —NC(NCN)NHR₉—, or —NC(NCN)NR₉R₁₀—;

A₂ is —O—, —S—, —SO—, —SO₂—, —CR₇R₈—, —CR₇R₈-A₃-, -A₃-CR₇R₈—, —NH—, —NR₉—, —NC(O)R₉—, —NSR₉—, —NSOR₉—, —NS(O)₂R₉—, —NC(S)R₉—, —NC(S)NHR₉—, —NC(S)NR₉R₁₀—, —NC(NH)NHR₉—, —NC(NH)NR₉R₁₀—, —NC(NCN)NHR₉—, or —NC(NCN)NR₉R₁₀—;

A₃ is —O—, —S—, —SO—, —SO₂—, —CR₇R₈—, —NH—, —NR₉—, —NC(O)R₉—, —NSR₉—, —NSOR₉—, —NS(O)₂R₉—, —NC(S)R₉—, —NC(S)NHR₉—, —NC(S)NR₉R₁₀—, —NC(NH)NHR₉—, —NC(NH)NR₉R₁₀—, —NC(NCN)NHR₉—, or —NC(NCN)NR₉R₁₀—;

Q₁ is —OR₇, —SR₇ or —NR₉R₁₀;

Q₂ is O, S, NH, or NR₉;

R₁ and R₂ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, —OS(O)₂OR₉, —OS(O)₂R₉, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₁ and R₂, together with the carbon atom to which each is attached, join to form a 3- to 9-membered carbocyclic or heterocyclic ring;

R₃, R₄, R₅, R₆, R₇, and R₈ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, —OS(O)₂OR₉, —OS(O)₂R₉, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring, or, for A₁, R₇ and R₈ together are oxo;

each R₉ and R₁₀ is, independently, —H, —C₁-C₈ alkyl, —C₃-C₁₂ cycloalkyl, —C₆-C₁₂ aryl, —C₇-C₁₄ arylalkyl, 3- to 9-membered heterocycle, —C₂-C₈ alkenyl, or —C₂-C₈ alkynyl;

each n is, independently, an integer ranging from 1 to 5;

m is an integer ranging from 0 to 3;

provided that A₁ is not —C(OH)₂— and/or or A₂ is not —C(OH)₂—;
that, when A₁ and A₂ are both —CH₂—, Q₁ is —NH₂, Q₂ is O, one of R₁ and R₂ is —H and the other is a —C₂-C₈ linear alkyl or a —C₄-C₈ branched alkyl, m=1 to 3, and

that A₁, A₂, and A₃ are not all —O—; and

any enantiomers, diastereomers, isomers, prodrugs and pharmaceutically acceptable salts thereof.

In certain specification embodiments, A₁ is C═O. In other embodiments, A₁ is not C═O.

In certain specific embodiments, A₁ is not —CH₂—.

In certain specific embodiments, A₁ is —O—.

In certain specific embodiments, A₁ is —S—.

In certain specific embodiments, A₁ is —NH—.

In certain specific embodiments, A₂ is not —CH₂—.

In certain specific embodiments, A₂ is —O—.

In certain specific embodiments, A₂ is —S—.

In certain specific embodiments, A₂ is —NH—.

In certain specific embodiments, A₂ is not —CR₇R₈-A₃- or -A₃-CR₇R₈—.

In certain specific embodiments, A₁ is —CH₂—, and A₂ is —CHF— or —CF₂—.

In certain specific embodiments, Q₁ is —NH₂, and Q₂ is O.

In certain specific embodiments, when present, R₉ or R₁₀ is unsubstituted phenyl.

In certain specific embodiments, R₁ and R₂ are both —H.

In certain specific embodiments, when present, R₉ and R₁₀ are independently —H or —CH₃.

In certain specific embodiments, R₁ and R₂ are not joined together with the carbon atom to which each is attached to form a 3- to 9-membered carbocyclic or heterocyclic ring.

In certain specific embodiments, R₃ and R₄, or R₃ and R₅, or R₃ and R₆, or R₄ and R₅, or R₄ and R₆, or R₅ and R₆, or R₇ and R₈, are not joined together with the carbon atom to which each is attached to form a 3- to 9-membered carbocyclic or heterocyclic ring.

The invention further provides compositions comprising a pharmaceutically acceptable carrier or vehicle and an effective amount of a compound having the Formula (Ia) as described above:

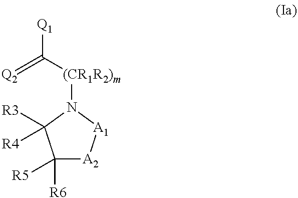

including enantiomers, diastereomers, isomers, prodrugs, or pharmaceutically acceptable salts thereof.

In another aspect, the invention provides methods for treating pain in a patient by administering to a patient in need thereof an effective amount of a compound of Formula (Ia), including enantiomers, diastereomers, isomers, prodrugs, pharmaceutically acceptable salts thereof, or pharmaceutically acceptable compositions thereof.

In still another aspect, the invention provides methods for treating inflammation in a patient by administering to a patient in need thereof an effective amount of a compound of Formula (Ia), including enantiomers, diastereomers, isomers, prodrugs, pharmaceutically acceptable salts thereof, or pharmaceutically acceptable compositions thereof.

In still another aspect, the invention provides methods for treating neuropathic pain in a patient by administering to a patient in need thereof an effective amount of a compound of Formula (Ia), including enantiomers, diastereomers, isomers, prodrugs, pharmaceutically acceptable salts thereof, or pharmaceutically acceptable compositions thereof.

In a further aspect, the present invention relates to methods useful for making compounds of Formula (Ia).

In one embodiment, the invention provides a method for making a compound of Formula (Ia):

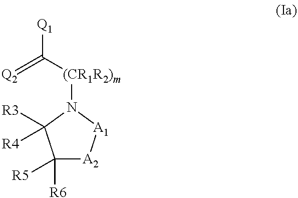

involving contacting a compound of Formula (IIa)

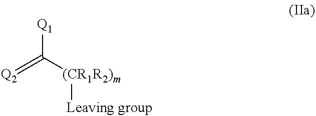

with a compound of Formula (IIIa)

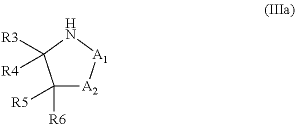

in the presence of an organic solvent and optionally a non-nucleophilic base, for a time and at a temperature sufficient to make the compound of Formula (Ia).

In a specific embodiment, a compound according to formula (Ia) is selected from the group consisting of:

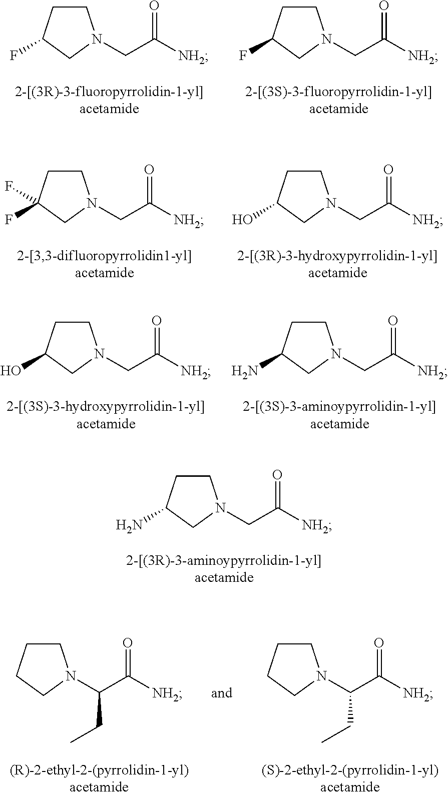

or pharmaceutically acceptable salts or compositions thereof.

In a specific embodiment, the compound having a structure according to formula (Ia) is

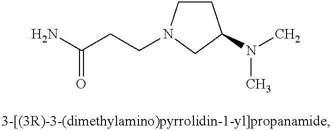

or a pharmaceutically acceptable salts or composition thereof.

In yet other embodiments, a piperidine compound having a structure according to formula (Ia) is selected from the group consisting of:

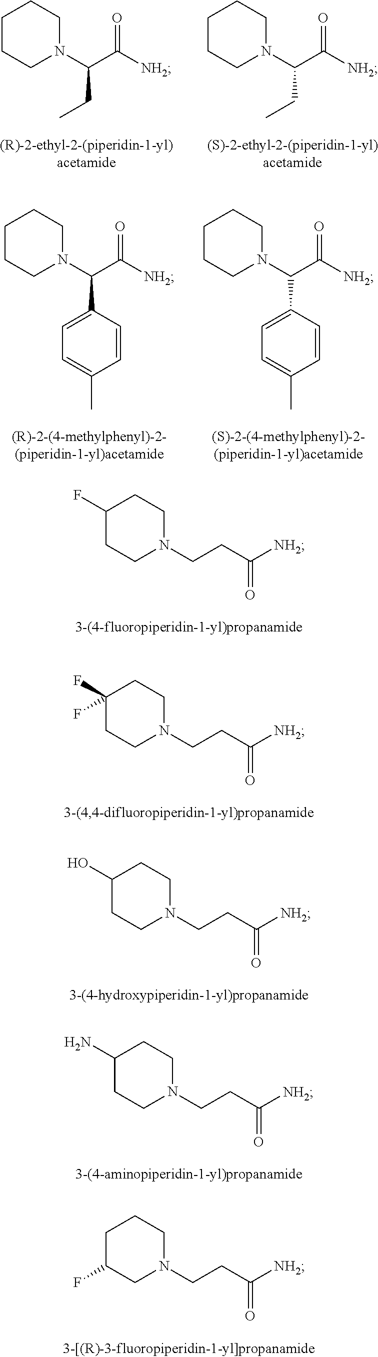

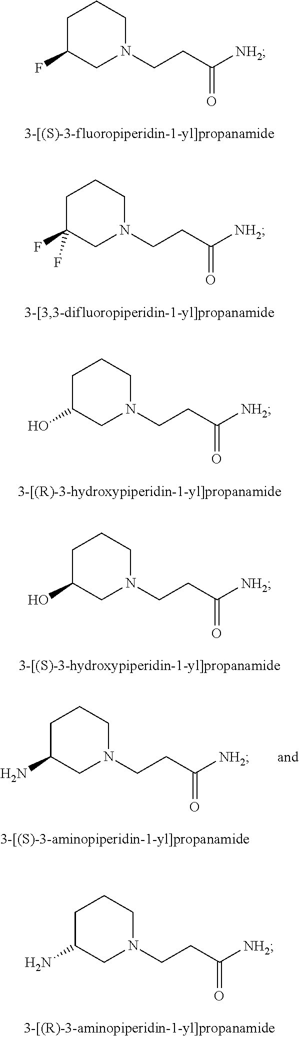

or pharmaceutically acceptable salts or compositions thereof.

In another aspect, the invention further provides methods for treating pain in a patient, comprising administering to a patient in need thereof an effective amount of a compound having the Formula (Ib), depicted below,

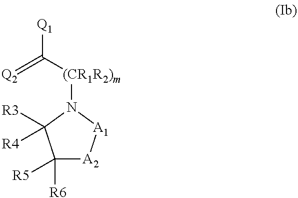

wherein:

A₁ is —CR₇R₈—;

A₂ is —CR₇R₈—, —CR₇R₈-A₃-, or -A₃-CR₇R₈—;

A₃ is —CR₇R₈—;

Q₁ is —OR₇, —SR₇ or —NR₉R₁₀;

Q₂ is O, S, NH, or NR₉;

R₁ and R₂ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, —OS(O)₂OR₉, —OS(O)₂R₉, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₁ and R₂ together with the carbon atom to which each is attached, join to form a 3- to 9-membered carbocyclic or heterocyclic ring;

R₃, R₄, R₅, R₆, R₇, and R₈ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, —OS(O)₂OR₉, —OS(O)₂R₉, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring, or, for A₁, R₇ and R₈ together are oxo;

each R₉ and R₁₀, are, independently, —H, —C₁-C₈ alkyl, —C₃-C₁₂ cycloalkyl, —C₆-C₁₂ aryl, —C₇-C₁₄ arylalkyl, 3- to 9-membered heterocycle, —C₂-C₈ alkenyl, or —C₂-C₈ alkynyl;

each n is, independently, an integer ranging from 1 to 5; and

m is an integer ranging from 0 to 3;

with the proviso that A₁ is not —C(OH)₂— and/or or A₂ is not —C(OH)₂—;

including enantiomers, diastereomers, isomers, prodrugs, pharmaceutically acceptable salts thereof, or pharmaceutically acceptable compositions thereof.

In certain specification embodiments, A₁ is C═O. In other embodiments, A₁ is not C═O.

In certain specific embodiments, A₁ is not —CH₂—.

In certain specific embodiments, A₂ is not —CH₂—.

In certain specific embodiments, A₂ is not —CR₇R₈-A₃- or -A₃-CR₇R₈—.

In certain specific embodiments, A₂ is —CH₂-A₃- or -A₃-CH₂—.

In certain specific embodiments, A₃ is —CH₂—.

In certain specific embodiments, A₁ and A₂ are —CH₂—.

In certain specific embodiments, when present, A₃ is —CHF— or —CF₂—.

In certain specific embodiments, A₁ is —CH₂—, and A₂ is —CHF— or —CF₂—.

In certain specific embodiments, Q₁ is —NH₂, and Q₂ is O.

In certain specific embodiments, when present, R₉ or R₁₀ is unsubstituted phenyl.

In certain specific embodiments, R₁ and R₂ are both —H.

In certain specific embodiments, when present, R₉ and R₁₀ are each, independently, —H or —CH₃.

In certain specific embodiments, R₁ and R₂ are not joined together with the carbon atom to which each is attached to form a 3- to 9-membered carbocyclic or heterocyclic ring.

In certain specific embodiments, R₃ and R₄, or R₃ and R₅, or R₃ and R₆, or R₄ and R₅, or R₄ and R₆, or R₅ and R₆, or R₇ and R₈, are not joined together with the carbon atom to which each is attached to form a 3- to 9-membered carbocyclic or heterocyclic ring.

In another aspect, the invention provides methods for treating inflammation in a patient, by administering to a patient in need thereof an effective amount of a compound of Formula (Ib), depicted above, including enantiomers, diastereomers, isomers, prodrugs, pharmaceutically acceptable salts thereof, or pharmaceutically acceptable compositions thereof.

In still another aspect, the invention provides methods for treating neuropathic pain in a patient, by administering to a patient in need thereof an effective amount of a compound of Formula (Ib), including enantiomers, diastereomers, isomers, prodrugs, pharmaceutically acceptable salts thereof, or pharmaceutically acceptable compositions thereof.

In a further aspect, the present invention relates to methods useful for making compounds having the Formula (Ib).

In one embodiment, the invention provides a method for making a compound having the Formula (Ib):

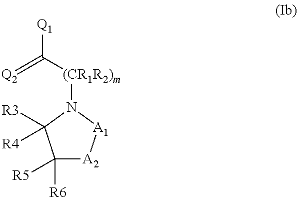

comprising contacting a compound of Formula (IIb)

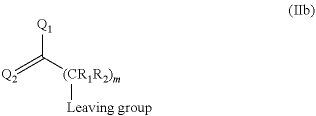

with a compound of Formula (IIIb)

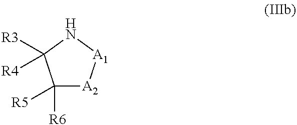

in the presence of an organic solvent and optionally a base, for a time and at a temperature sufficient to make the compound of Formula (Ib).

In a specific embodiment, a pyrrolidine compound according to Formula (Ib) is selected from the group consisting of:

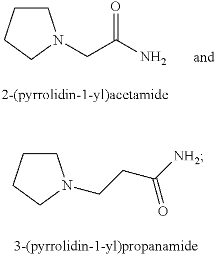

or pharmaceutically acceptable salts thereof.

In a specific embodiment, a piperidine compound according to Formula (Ib) is selected from the group consisting of:

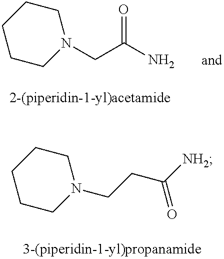

or pharmaceutically acceptable salts thereof.

The invention further features compounds of Formula (Ia) or (Ib) in which the nitrogen atom depicted in the formula is replaced with a carbon atom, e.g., as in 2-cyclopentylacetamide. The invention also features methods for use of these compounds in treating pain and/or inflammation, as described herein.

### DEFINITIONS AND ABBREVIATIONS

As used herein, “aldehyde” refers to a group having the structure represented by —C(O)H.

As used herein, “alkenyl” or “C₂-C₈ alkenyl” refers to an unsaturated, straight or branched chain hydrocarbon group containing 2-8 carbon atoms and at least one carbon-carbon double bond and which can be optionally substituted with a phenyl or naphthyl group.

As used herein, “alkyl” or “C₁-C₈ alkyl” refers to a straight or branched chain saturated hydrocarbon group containing 1-8 carbon atoms which can be optionally substituted with one or more -halogen, —NH₂, —OH, —O(C₁-C₈ alkyl), phenyl or naphthyl groups. Examples of C₁-C₈ straight or branched chain alkyl groups include, but are not limited to, methyl, ethyl, 1-propyl, 2-propyl, 1-butyl, 2-butyl, 2-methyl-1-propyl, 2-methyl-2-propyl, 1-pentyl, 2-pentyl, 3-pentyl, 2-methyl-1-butyl, 3-methyl-1-butyl, 2-methyl-3-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, 1-heptyl, and 1-octyl.

As used herein, “alkoxy” or “alkoxyl” refers to a group having the structure OR⁶, wherein R⁶ is selected from —C₁-C₈ alkyl, —C₂-C₈ alkenyl, —C₂-C₈ alkynyl, —C₃-C₁₂ cycloalkyl, —C₆-C₁₂ aryl, or —C₇-C₁₄ arylalkyl.

As used herein, “alkynyl” or “C₂-C₈ alkynyl” refers to an unsaturated, straight or branched chain hydrocarbon group containing 2-8 carbon atoms and at least one carbon-carbon triple bond and which can be optionally substituted with a phenyl or naphthyl group.

As used herein, “amido” refers to a group having the structure selected from —NR^{6a}R^{7a} or —C(O)NR⁶R⁷, wherein R^{6a} is selected from —C(O)R⁶, —C(O)NR⁶R⁷, and C(O)OR^{6a} and where R⁶, R⁷, and R^{7a} are, independently, selected from —H, —C₁-C₈ alkyl, —C₃-C₁₂ cycloalkyl, —C₆-C₁₂ aryl, —C₇-C₁₄ arylalkyl, 3- to 9-membered aromatic or non aromatic heterocycle, —C₂-C₈ alkenyl, —C₂-C₈ alkynyl, or R⁶ and R⁷ or R^{6a} and R^{7a}, together with the N atom to which each is attached, join to form a 3- to 7-membered aromatic or non aromatic heterocycle.

As used herein, “amino” refers to a group having the structure —NR⁶R⁷ wherein R⁶ and R⁷ are selected, independently, from —H, —C₁-C₈ alkyl, —C₃-C₁₂ cycloalkyl, —C₆-C₁₂ aryl, —C₇-C₁₄ arylalkyl, 3- to 9-membered aromatic or non aromatic heterocycle, —C₂-C₈ alkenyl, —C₂-C₈ alkynyl, or R⁶ and R⁷, together with the N atom to which each is attached, join to for a 3- to 7-membered heterocycle.

As used herein, “aromatic” refers to a cyclic ring system having (4n+2) π electrons in conjugation, where n is, for example, 1, 2, or 3, or 4.

As used herein, “C₆-C₁₂ aryl” refers to an optionally substituted monocyclic or bicyclic structure wherein all rings are aromatic and the rings are formed by carbon atoms. Exemplary aryl groups include phenyl and naphthyl.

As used herein, “arylalkyl” or “C₇-C₁₄ arylalkyl” refers to an optionally substituted group having the formula —(Cₓalkyl)-(C_{y}-aryl) wherein (x+y) is an integer between 7 and 14 and x is at least 1. Exemplary arylalkyls include benzyl and phenethyl.

As used herein, “carbocycle” refers to an optionally substituted C₃-C₁₂ monocyclic, bicyclic, or tricyclic structure in which the rings are formed only by carbon atoms. Carbocycles may be aromatic or may be non-aromatic.

As used herein, “carboxyl” or “carboxy” refers to a group having a structure selected from —C(O)R⁶, —OC(O)R⁶, —OC(O)OR⁶, —OC(O)NR⁶R⁷, —C(O)OR⁶, wherein R⁶ and R⁷ are, independently, selected from —H, —C₁-C₈ alkyl, —C₃-C₁₂ cycloalkyl, —C₆-C₁₂ aryl, —C₇-C₁₄ arylalkyl, 3- to 9-membered aromatic or non aromatic heterocycle, —C₂-C₈ alkenyl, and —C₂-C₈ alkynyl, or R⁶ and R⁷, together with the atom to which each is attached, join to form a 3- to 7-membered aromatic or non aromatic heterocycle;

As used herein, “carrier” or “pharmaceutical carrier” refers to a diluent, adjuvant, excipient, or vehicle with which a compound of the invention is administered. Such pharmaceutical carriers can be liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. The pharmaceutical carriers can be saline, gum acacia, gelatin, starch paste, talc, keratin, colloidal silica, urea, and the like. In addition, auxiliary, stabilizing, thickening, lubricating and coloring agents may be used. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, polyethylene glycol 300, water, ethanol, polysorbate 20, wetting or emulsifying agents, or pH buffering agents.

As used herein, “cyano” refers to a group having the structure —CN.

As used herein, “cycloalkyl” or “C₃-C₁₂ cycloalkyl” refers to an optionally substituted non-aromatic, saturated or partially unsaturated monocyclic, bicyclic or tricyclic hydrocarbon ring system containing 3-12 carbon atoms. Examples of C₃-C₁₂ cycloalkyl groups include but are not limited to cyclopropyl, cyclobutyl, cyclopentyl, cyclohexyl, cycloheptyl, cyclooctyl, norbornyl, adamantyl, bicyclo[2.2.2]oct-2-enyl, and bicyclo[2.2.2]octyl. Exemplary partially unsaturated cycloalkyls include cyclopropenyl, cyclobutenyl, cyclopentenyl, cyclopentadienyl, cyclohexenyl, cyclohexadienyl, cycloheptenyl, cycloheptadienyl, cycloheptatrienyl, cyclooctenyl, and cyclooctadienyl.

An “effective amount” is an amount of a compound that is effective for achieving the desired therapeutic effect. Representative, non-limiting examples of desirable therapeutic effects include treating or preventing pain, treating or preventing neuropathic pain, and treating or preventing inflammation. The effectiveness or effective amount of a compound for a given therapeutic use may be measured according to protocols known in the art, as exemplified in Example 2.

As used herein, “ester” refers to a carboxyl group having the structure —C(O)OR⁶, wherein R⁶ is selected from —C₁-C₈ alkyl, —C₃-C₁₂ cycloalkyl, —C₆-C₁₂ aryl, —C₇-C₁₄ arylalkyl, 3- to 9-membered aromatic or non aromatic heterocycle, —C₂-C₈ alkenyl, or —C₂-C₈ alkynyl.

As used herein, “haloalkyl” refers to an alkyl group wherein at least one hydrogen is replaced with a halogen. Haloalkyls may also be perhalogenated, as exemplified by trifluoromethyl.

As used herein, “halogen” refers to —F, —Cl, —Br, or —I.

As used herein, a “heterocycle” or “3- to 9-membered heterocycle” refers to an optionally substituted 3- to 9-membered aromatic or nonaromatic monocyclic or bicyclic ring, wherein the ring or rings comprise 1 to 4 heteroatoms selected from oxygen, nitrogen, and sulfur. Non-aromatic heterocycles may have one or more double bonds. Examples of double bonds include carbon-carbon double bonds (C═C), carbon-nitrogen double bonds (C═N), and nitrogen-nitrogen double bonds (N═N). Examples of nonaromatic 3- to 9-membered heterocycles include, but are not limited to, aziridinyl, oxiranyl, thiiranyl, azirinyl, diaziridinyl, diazirinyl, oxaziridinyl, azetidinyl, azetidinonyl, oxetanyl, thietanyl, piperidinyl, tetrahydropyridinyl, piperazinyl, morpholinyl, azepinyl or any partially or fully saturated derivatives thereof, diazepinyl or any partially or fully saturated derivatives thereof. Examples or aromatic 3- to 9-membered heterocycles include, but are not limited to, pyrrolyl, oxazinyl, thiazinyl, diazinyl, triazinyl, tetrazinyl, imidazolyl, benzimidazolyl, tetrazolyl, indolyl, isoquinolinyl, quinolinyl, quinazolinyl, pyrrolidinyl, purinyl, isoxazolyl, benzisoxazolyl, furanyl, furazanyl, pyridinyl, oxazolyl, benzoxazolyl, thiazolyl, benzthiazolyl, thiophenyl, pyrazolyl, triazolyl, benzodiazolyl, benzotriazolyl, pyrimidinyl, isoindolyl, and indazolyl.

As used herein, “heteroaryl” or “heteroaromatic” refers to an optionally substituted 3- to 9-membered heterocycle that is aromatic.

As used herein, “heterocycloalkyl” refers to a nonaromatic heterocycle.

As used herein, “hydroxy” refers to a group having the structure —OH.

As used herein, “imine” refers to a group having the structure —C(NR⁶) wherein R⁶ is selected from —H, —C₁-C₈ alkyl, —C₃-C₁₂ cycloalkyl, —C₆-C₁₂ aryl, —C₇-C₁₄ arylalkyl, 3- to 9-membered aromatic or non aromatic heterocycle, —C₂-C₈ alkenyl, and —C₂-C₈ alkynyl.

As used herein, “ketone” refers to a carboxyl group that has the structure —C(O)R⁶, wherein R⁶ is selected from —C₁-C₈ alkyl, —C₃-C₁₂ cycloalkyl, —C₆-C₁₂ aryl, —C₇-C₁₄ arylalkyl, 3- to 9-membered aromatic or non aromatic heterocycle, —C₂-C₈ alkenyl, and —C₂-C₈ alkynyl.

As used herein, “lower alkenyl” refers to an optionally substituted C₂-C₅ straight or branched alkenyl group.

As used herein, “lower alkyl” refers to an optionally substituted C₁-C₅ straight or branched alkyl group which can be unsubstituted or optionally substituted with one or more -halogen, —NH₂, —OH, —O(C₁-C₈-alkyl), phenyl or naphthyl groups. Examples of optionally substituted C₁-C₅ straight or branched chain alkyl groups include, but are not limited to, methyl, ethyl, 1-propyl, 2-propyl, 1-butyl, 2-butyl, 2-methyl-1-propyl, 2-methyl-2-propyl, 1-pentyl, 2-pentyl, 3-pentyl, 2-methyl-1-butyl, 3-methyl-1-butyl, 2-methyl-3-butyl, 2,2-dimethyl-1-propyl and 1-pentyl.

As used herein, “lower alkynyl” refers to an optionally substituted C₂-C₅ straight or branched alkynyl group.

As used herein, “non-aromatic carbocycle” refers to an optionally substituted monocyclic, bicyclic, or tricyclic structure wherein the atoms that form the ring are all carbons and at least one ring does not have 4n+2π electrons in conjugation. Carbocycles include cycloalkyls, partially unsaturated cycloalkyls, or an aromatic ring fused to a cycloalkyl or fused to a partially unsaturated cycloalkyl. In addition to cycloalkyls, and partially unsaturated cycloalkyls, exemplary non-aromatic carbocycles include tetrahydronaphthyl.

By “oxo” is meant a group having a structure ═O, wherein an oxygen atom makes a double bond to another element such as C, S, or P.

As used herein, “pharmaceutically acceptable” means approved by a regulatory agency of the Federal or a state government or listed in the U.S. Pharmacopeia or other generally recognized pharmacopeia for use in animals, and more particularly in humans.

As used herein, “pharmaceutically acceptable salt(s)” include but are not limited to salts of acidic or basic groups that may be present in compounds used in the present compositions. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, mesylate, hydroxymethylsulfonate, hydroxyethyl sulfonate, and pamoate (i.e., 1,1′-methylene-bis-(2-hydroxy-3-naphthoate)) salts.

As used herein, “phosphine” refers to a group having the structure —P(R^{6a})(R^{6b}), wherein R^{6a} and R^{6b} are selected, independently, from —H, —C₁-C₈ alkyl, —C₃-C₁₂ cycloalkyl, —C₆-C₁₂ aryl, —C₇-C₁₄arylalkyl, 3- to 9-membered aromatic or non aromatic heterocycle, —C₂-C₈ alkenyl, and —C₂-C₈ alkynyl or R^{6a} and R^{6b}, together with the P-atom to which each is attached, join to form a 3- to 7-membered ring.

As used herein, “phosphonato” refers to a group having the structure —PO(OR⁶)(OR⁷), wherein R⁶ and R⁷ are, independently, —H, —C₁-C₈ alkyl, —C₃-C₁₂ cycloalkyl, —C₆-C₁₂ aryl, —C₇-C₁₄ arylalkyl, 3- to 9-membered aromatic or non aromatic heterocycle, —C₂-C₈ alkenyl, and —C₂-C₈ alkynyl or R⁶ and R⁷, together with the atom to which each is attached, join to form a 3- to 7-membered ring.

As used herein, “sulfonamide” refers to a group having a structure selected from —S(O)NR⁶R⁷ or —S(O)₂NR⁶R⁷, wherein R⁶ and R⁷ are, independently, —H, —C₁-C₈ alkyl, —C₃-C₁₂ cycloalkyl, —C₆-C₁₂ aryl, —C₇-C₁₄ arylalkyl, 3- to 9-membered aromatic or non aromatic heterocycle, —C₂-C₈ alkenyl, and —C₂-C₈ alkynyl or R₆ and R₇, together with the N-atom to which each is attached, join to form a 3- to 7-membered heterocycle.

As used herein, “sulfonyl” refers to a group having a structure selected from —S(O)R⁶, and —S(O)₂R⁶, wherein R⁶ is selected from —C₁-C₈ alkyl, —C₃-C₁₂ cycloalkyl, —C₆-C₁₂ aryl, —C₇-C₁₄arylalkyl, 3- to 9-membered aromatic or non aromatic heterocycle, —C₂-C₈ alkenyl, or —C₂-C₈ alkynyl.

As used herein, “thiocarbonyl” or “thiocarboxy” refers to a group having a structure selected from —C(S)R⁶, —OC(S)R⁶, —OC(S)OR⁶, —OC(S)NR⁶R⁷, —C(S)NR₆R₇, —C(S)OR⁶, wherein R⁶ and R⁷ are, independently, selected from —H, —C₁-C₈ alkyl, —C₃-C₁₂ cycloalkyl, —C₆-C₁₂ aryl, —C₇-C₁₄ arylalkyl, 3- to 9-membered aromatic or non aromatic heterocycle, —C₂-C₈ alkenyl, and —C₂-C₈ alkynyl or R⁶ and R⁷, together with the atom to which each is attached, join to form a 3- to 7-membered aromatic or non aromatic carbocycle or heterocycle;

As used herein, “thioether” refers to a group having the structure —SR⁶, wherein R⁶ is selected from —C₁-C₈ alkyl, —C₃-C₁₂ cycloalkyl, —C₆-C₁₂ aryl, —C₇-C₁₄ arylalkyl, 3- to 9-membered aromatic or non aromatic heterocycle, —C₂-C₈ alkenyl, or —C₂-C₈ alkynyl.

As used herein, “thiol” refers to a group having the structure —SH.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diasteroemers of the compound. A typical stereomerically pure compound includes greater than about 80% by weight of stereoisomer of the compound and less than about 20% by weight of other stereoisomers the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

When the groups described herein are said to be “substituted or unsubstituted” or “optionally substituted,” when substituted, they may be substituted with any desired substituent or substituents that do not adversely affect the desired activity of the compound. Examples of preferred substituents are those found in the exemplary compounds and embodiments disclosed herein, as well as halogen (chloro, iodo, bromo, or fluoro); C₁₋₆ alkyl; C₂₋₆ alkenyl; C₂₋₆ alkynyl; hydroxyl; C₁₋₆ alkoxyl; amino; nitro; thiol; thioether; imine; cyano; amido; phosphonato; phosphine; carboxyl; thiocarbonyl; sulfonyl; sulfonamide; ketone; aldehyde; ester; oxygen (═O); haloalkyl (e.g., trifluoromethyl); cycloalkyl, which may be monocyclic (e.g., cyclopropyl, cyclobutyl, cyclopentyl, or cyclohexyl) or fused or non-fused polycyclic, or a heterocycloalkyl, which may be monocyclic or fused or non-fused polycyclic (e.g., pyrrolidinyl, piperidinyl, piperazinyl, morpholinyl, or thiazinyl); carbocyclic or heterocyclic, monocyclic or fused or non-fused polycyclic aryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl); benzyloxy; amino(primary, secondary, or tertiary); —N(CH₃)₂; O-lower alkyl; O-aryl; aryl-lower alkyl; CO₂CH₃; —OCH₂CH₃; methoxy; CONH₂; OCH₂CONH₂; NH₂; SO₂NH₂; OCHF₂; CF₃; or OCF₃. These substituents may optionally be further substituted with a substituent selected from such groups or may also be optionally substituted by a fused-ring structure or bridge, for example —OCH₂O—.

The phrase “substantially anhydrous,” as used herein in connection with a reaction mixture or an organic solvent, means that the reaction mixture or organic solvent comprises less than about 1 percent of water by weight; in one embodiment, less than about 0.5 percent of water by weight; and in another embodiment, less than about 0.25 percent of water by weight of the reaction mixture or organic solvent.

In one embodiment, when administered to a patient, e.g., a mammal for veterinary use or a human for clinical use, the compounds of the invention are administered in isolated form.

As used herein, “isolated” means that the compounds of the invention are separated from other components of either (a) a natural source, such as a plant or cell, preferably bacterial culture, or (b) a synthetic organic chemical reaction mixture. In another embodiment, via conventional techniques, the compounds of the invention are purified. As used herein, “purified” means that when isolated, the isolate contains at least 95%, preferably at least 98%, of a single compound of the invention by weight of the isolate.

It is recognized that compounds of the invention can have one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding enantiomers and stereoisomers, that is, both the stereomerically pure form (e.g., geometrically pure, enantiomerically pure, or diastereomerically pure) and enantiomeric and stereoisomeric mixtures, e.g., racemates.

Enantiomeric and stereoisomeric mixtures of compounds of the invention can usually be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

It should be noted that if there is a discrepancy between a depicted structure and a name given that structure, the depicted structure controls. In addition, if the stereochemistry of a structure or a portion of a structure is not indicated with, for example, bold or dashed lines, the structure or portion of the structure is to be interpreted as encompassing all stereoisomers of it.

The following abbreviations and their definitions, unless defined otherwise, are used in this specification:

| Abbreviation | Definition |
|---|---|
| BOC | —C(O)OC(CH₃)₃ |
| DEF | N,N-diethylformamide |
| DMF | N,N-dimethylformamide |
| DMSO | dimethylsulfoxide |
| THF | tetrahydrofuran |
| EtOAc | ethyl acetate |
| EtOH | ethanol |
| MeOH | methanol |
| Tf | —SO₂CF₃ |
| dba | dibenzylideneacetone |
| Ph | Phenyl |
| TBDMSCl | tert-Butyldimethylsilyl chloride |
| DBU | 1,8-diazabicyclo[5.4.0]undec-7-ene |
| LC/MS | Liquid Chromatography/Mass |
| | Spectrometry |

### DETAILED DESCRIPTION OF THE INVENTION

### Compounds of Formula (IA)

As stated above, the present invention provides compounds having the Formula (Ia)

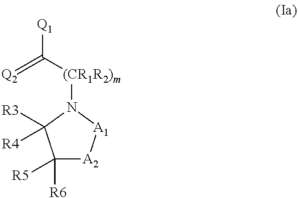

including enantiomers, diastereomers, isomers, prodrugs and pharmaceutically acceptable salts thereof, wherein:

Q₁, Q₂, A₁, A₂, m, R₁, R₂, and R₃ to R₆ are defined above for the compounds of formula (Ia).

A first subclass of the compounds of Formula (Ia) is that wherein:

A₁ is —O—, —S—, —SO—, —SO₂—, —CR₇R₈, —NH—, —NR₉—, —NC(O)R₉—, —NSR₉—, —NSOR₉—, —NS(O)₂R₉—, —NC(S)R₉—, —NC(S)NHR₉—, —NC(S)NR₉R₁₀—, —NC(NH)NHR₉, —NC(NH)NR₉R₁₀—, —NC(NCN)NHR₉—, or —NC(NCN)NR₉R₁₀—;

A₂ is —O—, —S—, —SO—, —SO₂—, —CR₇R₈, —CR₇R₈-A₃-, -A₃-CR₇R₈—, —NH—, —NR₉—, —NC(O)R₉—, —NSR₉—, —NSOR₉—, —NS(O)₂R₉—, —NC(S)R₉—, —NC(S)NHR₉—, —NC(S)NR₉R₁₀—, —NC(NH)NHR₉—, —NC(NH)NR₉R₁₀—, —NC(NCN)NHR₉—, or —NC(NCN)NR₉R₁₀—;

A₃ is —O—, —S—, —SO—, —SO₂—, —CR₇R₈, —NH—, —NR₉—, —NC(O)R₉—, —NSR₉—, —NSOR₉—, —NS(O)₂R₉—, —NC(S)R₉—, —NC(S)NHR₉—, —NC(S)NR₉R₁₀—, —NC(NH)NHR₉—, —C(NH)NR₉R₁₀—, —NC(NCN)NHR₉—, or —NC(NCN)NR₉R₁₀—;

Q₁ is —NR₉R₁₀;

Q₂ is O;

R₁ is —H;

R₂ is —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —NHC(O)R₉, —NHSR₉, —NHSOR₉, —NHS(O)₂R₉, —OS(O)₂O⁻, —OS(O)₂R₉, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, or —NR₉C(NCN)NR₉R₁₀, or R₁ and R₂ together with the carbon atom to which each is attached, join to form a 3- to 9-membered ring;

R₃, R₄, R₅, R₆, R₇, and R₈ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀ or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;

R₉ and R₁₀ are each, independently, —H, —CH₃, —CH₂CH₃, —CH(CH₃)₂, -phenyl, or -benzyl;

each n is, independently, an integer ranging from 1 to 2;

m is an integer ranging from 0 to 3;

with the proviso that A₁ and A₂ are not —C(OH)₂—;
with the added proviso that when A₁ and A₂ are simultaneously —CH₂—, Q₁ is —NH₂, Q₂ is O, R₁ is H, R₂ is a —C₂-C₈ linear alkyl or a —C₄-C₈ branched alkyl, m=1 to 3;
with a further proviso that A₁, A₂, and A₃ are not all —O—.

A second subclass of the compounds of Formula (Ia) is that wherein:

A₁ is —O—, —S—, —SO—, —SO₂—, —CR₇R₈, —NH—, —NR₉—, —NC(O)R₉—, —NS(O)₂R₉—, —NC(NH)NHR₉—, —NC(NH)NR₉R₁₀—, —NC(NCN)NHR₉—, or —NC(NCN)NR₉R₁₀—;

A₂ is —O—, —S—, —SO—, —SO₂—, —CR₇R₈, —CR₇R₈-A₃-, -A₃-CR₇R₈—, —NH—, —NR₉—, —NC(O)R₉—, —NS(O)₂R₉—, —NC(S)R₉—, —NC(NH)NHR₉—, —NC(NH)NR₉R₁₀—, —NC(NCN)NHR₉—, or —NC(NCN)NR₉R₁₀—;

A₃ is —O—, —S—, —SO—, —SO₂—, —CR₇R₈, —NH—, —NR₉—, —NC(O)R₉—, —NS(O)₂R₉—, —NC(NH)NHR₉—, —NC(NH)NR₉R₁₀—, —NC(NCN)NHR₉—, or —NC(NCN)NR₉R₁₀—;

R₁ is —H;

R₂ is —H, —OH, halogen, —CN, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)NR₉R₁₀, —C(O)OR₉, —SR₉, —SOR₉, —S(O)₂R₉, —NHC(O)R₉, —NHS(O)₂R₉, —NHC(NH)NHR₉, —NR₉C(NH)NHR₉, —NHC(NH)NR₉R₁₀, —NR₉C(NH)NR₉R₁₀, —NHC(NCN)NHR₉, or —NR₉C(NCN)NR₉R₁₀, or R₁ and R₂ together with the carbon atom to which each is attached, join to form a 3- to 9-membered carbocyclic or heterocyclic ring;

R₃, R₄, R₅, R₆, R₇, and R₈ are, independently, —H, —OH, halogen, —CN, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;

each R₉ and R₁₀ are, independently, —H, —C₁-C₈ alkyl, —C₃-C₁₂ cycloalkyl, —C₆-C₁₂ aryl, or —C₇-C₁₄ arylalkyl; and

m is an integer ranging from 1 to 3; with the proviso that A₁ and A₂ are not —C(OH)₂—;

with the added proviso that when A₁ and A₂ are simultaneously —CH₂—, Q₁ is —NH₂, Q₂ is O, one of R₁ and R₂ is H and the other is a —C₂-C₈ linear alkyl or a —C₄-C₈ branched alkyl, m=1 to 3;

with a further proviso that A₁, A₂, and A₃ are not all —O—.

A third subclass of the compounds of Formula (Ia) is that wherein:

A₁ is —O—, —S—, —SO—, —SO₂—, —CR₇R₈—, —NH—, —NR₉—, —NC(O)R₉—, or —NS(O)₂R₉—;

A₂ is —O—, —S—, —SO—, —SO₂—, —CR₇R₈—, —CR₇R₈-A₃-, -A₃-CR₇R₈—, —NH—, —NR₉—, —NC(O)R₉—, or —NS(O)₂R₉—;

A₃ is —O—, —S—, —SO—, —SO₂—, —CR₇R₈—, —NH—, —NR₉—, —NC(O)R₉—, or —NS(O)₂R₉—;

R₁ is —H;

R₂ is —H, —OH, halogen, —CN, —C₁-C₄ alkyl, —C₂-C₆ alkenyl, —C₂-C₆ alkynyl, —C₃-C₅ cycloalkyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)NR₉R₁₀, —C(O)OR₉, —SR₉, —SOR₉, —S(O)₂R₉, —NHC(O)R₉, or —NHS(O)₂R₉, or R₁ and R₂ together with the carbon atom to which each is attached, join to form a 3- to 5-membered carbocyclic or heterocyclic ring;

R₃, R₄, R₅, R₆, R₇, and R₈ are, independently, —H, —OH, halogen, —CN, —SH, —N₃, —C₁-C₄ alkyl, —C₂-C₆ alkenyl, —C₂-C₆ alkynyl, —C₃-C₅ cycloalkyl, -phenyl, —C₇-C₁₀ arylalkyl, 3 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 6-membered carbocyclic or heterocyclic ring;

each R₉ and R₁₀ are, independently, —H, —C₁-C₄ alkyl, —C₃-C₆ cycloalkyl, phenyl, or —C₇-C₁₀ arylalkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, Q₂ is O, R₁ is H, R₂ is a —C₂-C₈ linear alkyl or a —C₄-C₈ branched alkyl, m=1 to 3;

with a further proviso that A₁, A₂, and A₃ are not all —O—.

A fourth subclass of the compounds of Formula (Ia) is that wherein:

A₁ is —O—, —S—, —SO₂—, —CR₇R₈—, —NH—, or —NR₉—;

A₂ is —O—, —S—, —SO₂—, —CR₇R₈—, —CR₇R₈-A₃-, -A₃-CR₇R₃—, —NH—, or —NR₉—;

A₃ is —O—, —S—, —SO₂—, —CR₇R₈, —NH—, or —NR₉—;

Q₁ is —OR₇ or —NR₉R₁₀;

Q₂ is O;

R₁ is —H;

R₂ is —H, —C₁-C₄ alkyl, or —C₂-C₆ alkenyl, or R₁ and R₂ together with the carbon atom to which each is attached, join to form a 3- to 5-membered carbocyclic or heterocyclic ring;

R₃, R₄, R₅, R₆, R₇, and R₈ are, independently, —H, —OH, halogen, —CN, —SH, —N₃, —C₁-C₄ alkyl, —C₂-C₆ alkenyl, —C₂-C₆ alkynyl, —C₃-C₅ cycloalkyl, -phenyl, -benzyl, 3 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 6-membered carbocyclic or heterocyclic ring;

each R₉ and R₁₀ are, independently, —H, —C₁-C₄ alkyl, —C₃-C₆ cycloalkyl, -phenyl, or -benzyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, Q₂ is O, R₁ is H, and R₂ is a —C₂-C₄ linear alkyl or a —C₄-C₆ branched alkyl, m=1 to 2;

with a further proviso that A₁, A₂, and A₃ are not all —O—.

A fifth subclass of the compounds of Formula (Ia) is that wherein:

A₁ is —O—, —S—, —SO₂—, —CR₇R₈—, —NH—, or —NR₉—;

A₂ is —O—, —S—, —SO₂—, —CR₇R₈—, —CR₇R₈-A₃-, -A₃-CR₇R₈—, —NH—, or —NR₉—;

A₃ is —O—, —S—, —SO₂—, —CR₇R₈—, —NH—, or —NR₉—;

Q₁ is —OR₇ or —NR₉R₁₀;

Q₂ is O;

R₁ is —H;

R₂ is —H, or —C₁-C₄ alkyl;

R₃, R₄, R₅, and R₆ are, independently, —H, —OH, —F, —Cl, —CN, —C₁-C₄ alkyl, —C₂-C₆ alkenyl, -phenyl, -benzyl, 5 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, together with the carbon atom to which each is attached, join to form a 5- to 6-membered carbocyclic or heterocyclic ring;

R₇ and R₈ are, independently, —H, —OH, halogen, —CN, —SH, —N₃, —C₁-C₄ alkyl, —C₂-C₆ alkenyl, —C₂-C₆ alkynyl, —C₃-C₅ cycloalkyl, -phenyl, -benzyl, 5 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₇ and R₈ together with the carbon atom to which each is attached, join to form a 5- to 6-membered carbocyclic or heterocyclic ring;

each R₉ and R₁₀ are, independently, —H, —C₁-C₄ alkyl, —C₃-C₆ cycloalkyl, -phenyl, or -benzyl;

m is an integer ranging from 1 to 2;

with the proviso that A₁ and A₂ are not —C(OH)₂—;

with the added proviso that when A₁ and A₂ are simultaneously —CH₂—, then Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not simultaneously —O—.

A sixth subclass of the compounds of Formula (Ia) is that wherein:

A₁ is —O—, —S—, —SO₂—, —CR₇R₈—, —NH—, or —NR₉—;

A₂ is —O—, —S—, —SO₂—, —CR₇R₈—, —CR₇R₈-A₃-, -A₃-CR₇R₈—, —NH—, or —NR₉—;

A₃ is —O—, —S—, —SO₂—, —CR₇R₈—, —NH—, or —NR₉—;

Q₁ is —OR₇ or —NR₉R₁₀;

Q₂ is O;

R₁ is —H;

R₂ is —H, or —C₁-C₄ alkyl;

R₃, R₄, R₅, and R₆ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆ together with the carbon atom to which each is attached, join to form a 5- to 6-membered carbocyclic or heterocyclic ring;

R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₇ and R₈, together with the carbon atom to which each is attached, join to form a 5- to 6-membered carbocyclic or heterocyclic ring;

each R₉ and R₁₀ are, independently, —H, —C₁-C₄ alkyl, —C₃-C₆ cycloalkyl, -phenyl, or -benzyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not all —O—.

A seventh subclass of the compounds of Formula (Ia) is that wherein:

A₁ is —O—, —S—, —SO₂—, —CR₇R₈—, —NH—, or —NR₉—;

A₂ is —O—, —S—, —SO₂—, —CR₇R₈—, —CR₇R₈-A₃-, -A₃-CR₇R₈—, —NH—, or —NR₉—;

A₃ is —O—, —S—, —SO₂—, —CR₇R₈—, —NH—, or —NR₉—;

Q₁ is —OR₇ or —NR₉R₁₀;

Q₂ is O;

R₁ is —H;

R₂ is —H, or —C₁-C₄ alkyl;

R₃, R₄, R₅, and R₆ are, independently, —H, —OH, —F, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₅, or R₃ and R₆, or R₄ and R₅, or R₄ and R₆, together with the carbon atom to which each is attached, join to form a 5- to 6-membered carbocyclic or heterocyclic ring;

R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not all —O—.

An eighth subclass of the compounds of Formula (Ia) is that wherein:

A₁ is —O—, —S—, —SO₂—, —CR₇R₈—, —NH—, or —NR—;

A₂ is —O—, —S—, —SO₂—, —CR₇R₈—, —CR₇R₈-A₃-, -A₃-CR₇R₈—, —NH—, or —NR₉—;

A₃ is —O—, —S—, —SO₂—, —CR₇R₈—, —NH—, or —NR₉—;

Q₁ is —OR₇ or —NR₉R₁₀;

Q₂ is O;

R₁ is H;

R₂ is —H, or —C₁-C₄ alkyl;

R₃, R₄, R₅, and R₆ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not all —O—.

A ninth subclass of the compounds of Formula (Ia) is that wherein:

A₁ is —O—, —S—, —SO₂—, —CR₇R₈—, —NH—, or —NR₉—;

A₂ is —O—, —S—, —SO₂—, —CR₇R₈—, —CR₇R₈-A₃-, -A₃-CR₇R₈—, —NH—, or —NR₉—;

A₃ is —O—, —S—, —SO₂—, —CR₇R₈—, —NH—, or —NR₉—;

Q₁ is —OR₇;

Q₂ is O;

R₁ is —H;

R₂ is —H, or —C₁-C₄ alkyl;

R₃, R₄, R₅, and R₆ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

R₇ and R₈ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not all —O—.

A tenth subclass of the compounds of Formula (Ia) is that wherein:

A₁ is —O—, —S—, —SO₂—, —CR₇R₈—, —NH—, or —NR₉—;

A₂ is —O—, —S—, —SO₂—, —CR₇R₈—, —CR₇R₈-A₃-, -A₃-CR₇R₈—, —NH—, or —NR₉—;

A₃ is —O—, —S—, —SO₂—, —CR₇R₈—, —NH—, or —NR₉—;

Q₁ is —NR₉R₁₀;

Q₂ is O;

R₁ is —H;

R₂ is —H, or —C₁-C₄ alkyl;

R₃, R₄, R₅, and R₆ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

R₇ and R₈ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not all —O—.

An eleventh subclass of the compounds of Formula (Ia) is that wherein:

A₁ is —O—, —S—, —CR₇R₈—, or —NR₉—;

A₂ is —CR₇R₈—, —CR₇R₈-A₃-, or -A₃-CR₇R₈—;

A₃ is —CR₇R₈—;

Q₁ is —NR₉R₁₀;

Q₂ is O;

R₁ is H;

R₂ is —H, or —C₁-C₄ alkyl;

R₃, R₄, R₅, and R₆ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC—OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

R₇ and R₈ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein; and

with a further proviso that A₁, A₂ and A₃ are not all —O—.

Further subsets within the eleventh subclass of the compounds of Formula (Ia) are those wherein:

A₁ is —O—;

A₁ is —S—;

A₁ is —CR₇R₈—;

A₁ is —NR₉—;

A₂ is —CR₇R₈—, with the proviso that A₁ is not —C(OH)₂—, and with the added proviso that when A₁ and A₂ are all —CH₂—, Q₁ is —NH₂ and R₂ is a —C₂-C₄ linear alkyl or a —C₄ branched alkyl;

A₂ is —CR₇R₈-A₃- or -A₃-CR₇R₃—, with the proviso that A₁ is not —C(OH)₂—.

A twelfth subclass of the compounds of Formula (Ia) is that wherein:

A₁ is —CR₇R₈—;

A₂ is —O—, —S—, —NR₉—, —CR₇R₈—, —CR₇R₈-A₃-, or -A₃-CR₇R₈—;

A₃ is —CR₇R₈—;

Q₁ is —NR₉R₁₀;

Q₂ is O;

R₁ is —H;

R₂ is —H, or —C₁-C₄ alkyl;

R₃, R₄, R₅, and R₆ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

R₇ and R₈ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are all —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂ and A₃ are not all —O—.

Further subsets within the twelfth subclass of the compounds of Formula (Ia) are those wherein:

A₂ is —O—, with the proviso that A₁ is not —C(OH)₂—;

A₂ is —S—, with the proviso that A₁ is not —C(OH)₂—;

A₂ is —CR₇R₈—, with the proviso that A₁ is not —C(OH)₂— and with the added proviso that when A₁ and A₂ are both —CH₂— that Q₁ is —NH₂, and R₂ is a —C₂-C₄ linear alkyl or a —C₄ branched alkyl;

A₂ is —NR₉—, with the proviso that A₁ is not —C(OH)₂—;

A₂ is —CR₇R₈—, with the proviso that A₁ is not —C(OH)₂—; and

A₂ is -A₃-CR₇R₈—, with the proviso that A₁ is not —C(OH)₂—.

A thirteenth subclass of the compounds of Formula (Ia) is that wherein:

A₁ is —CR₇R₈—;

A₂ is —CR₇R₈—, —CR₇R₈-A₃-, or -A₃-CR₇R₈—;

A₃ is —O—, —S—, —NR₉—, or —CR₇R₃—;

Q₁ is —NR₉R₁₀;

Q₂ is O;

R₁ is —H;

R₂ is —H or —C₁-C₄ alkyl;

R₃, R₄, R₅, and R₆ are, independently, —H, —OH, F, Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

R₇ and R₈ are, independently, —H, —OH, F, Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

Each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not all —O—.

Further subsets within the thirteenth subclass of the compounds of Formula (Ia) are those wherein:

A₃ is —O—, with the proviso that A₁ is not —C(OH)₂—;

A₃ is —S—, with the proviso that A₁ is not —C(OH)₂—;

A₃ is —CR₇R₈—, with the proviso that A₁ is not —C(OH)₂—; and

A₃ is —NR₉—, with the proviso that A₁ is not —C(OH)₂—.

A fourteenth subclass of the compounds of Formula (Ia) is that wherein:

A₁ is —O—, —S—, —SO₂—, —CR₇R₈—, —NH—, or —NR₉—;

A₂ is —O—, —S—, —SO₂—, —CR₇R₈—, —CR₇R₈-A₃-, -A₃-CR₇R₈—, —NH—, or —NR₉—;

A₃ is —O—, —S—, —SO₂—, —CR₇R₈—, —NH—, or —NR₉—;

Q₁ is —OR₇ or —NR₉R₁₀;

Q₂ is O;

R₁ is —H;

R₂ is —H or —C₁-C₄ alkyl;

R₃ and R₅, or R₃ and R₆, or R₄ and R₅, or R₄ and R₆, together with the carbon atom to which each is attached, join to form a 5- to 6-membered carbocyclic or heterocyclic ring;

R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not all —O—.

A fifteenth subclass of the compounds of Formula (Ia) is that wherein:

A₁ is —O—, —S—, —CR₇R₈—, or —NR₉—;

A₂ is —S—, —CR₇R₈—, —CR₇R₈-A₃-, -A₃-CR₇R₈—, or —NR₉—;

A₃ is —O—, —S—, CR₇R₈—, or —NR₉—,

Q₁ is —OR₇ or —NR₉R₁₀;

Q₂ is O;

R₁ is —H;

R₂ is —H, or —C₁-C₄ alkyl;

R₃ and R₅, or R₃ and R₆, or R₄ and R₅, or R₄ and R₆, together with the carbon atom to which each is attached, join to form a 5- to 6-membered carbocyclic or heterocyclic ring;

R₇ and R₈ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not all —O—.

An sixteenth subclass of the compounds of Formula (Ia) is that wherein:

A₁ is —O—, —S—, —CR₇R₈—, or —NR₉—;

A₂ is —O—, —S—, —CR₇R₈—, —CR₇R₈-A₃-, -A₃-CR₇R₈—, or —NR₉—;

A₃ is —O—, —S—, CR₇R₈—, or —NR₉—;

Q₁ is —NR₉R₁₀;

Q₂ is O;

R₁ is —H;

R₂ is —H or —C₁-C₄ alkyl;

R₃ and R₅, or R₃ and R₆, or R₄ and R₅, or R₄ and R₆, together with the carbon atom to which each is attached, join to form a 5- to 6-membered carbocyclic or heterocyclic ring;

R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)_{OR}—C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein; and

with a further proviso that A₁, A₂, and A₃ are not all —O—.

A seventeenth subclass of the compounds of Formula (Ia) is that wherein:

A₁ is —O—, —S—, —CR₇R₈—, or —NR₉—;

A₂ is —CR₇R₈—, —CR₇R₈-A₃-, or -A₃-CR₇R₅—;

A₃ is —CR₇R₈—;

Q₁ is —NR₉R₁₀;

Q₂ is O;

R₁ is —H;

R₂ is —H, or —C₁-C₄ alkyl;

R₃ and R₅, or R₃ and R₆, or R₄ and R₅, or R₄ and R₆, together with the carbon atom to which each is attached, join to form a 5- to 6-membered carbocyclic or heterocyclic ring;

R₇ and R₈ are, independently, —H, —OH, —F, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not all —O—.

Further subsets within the seventeenth subclass of the compounds of Formula (Ia) are those wherein:

A₁ is —O—;

A₁ is —S—;

A₁ is —CR₇R₈—;

A₁ is —NR₉—;

A₂ is —CR₇R₈—, with the added proviso that when A₁ and A₂ are both —CH₂—, Q₁ is —NH₂, and R₂ is a —C₂-C₄ linear alkyl or a —C₄ branched alkyl; and

A₂ is —CR₇R₈-A₃- or -A₃-CR₇R₈—.

An eighteenth subclass of the compounds of Formula (Ia) is that wherein:

A₁ is —CR₇R₈—;

A₂ is —O—, —S—, —NR₉—, —CR₇R₈—, —CR₇R₈-A₃-, or -A₃-CR₇R₈—;

A₃ is —CR₇R₈—;

Q₁ is —NR₉R₁₀;

Q₂ is O;

R₁ is —H;

R₂ is —H, or —C₁-C₄ alkyl;

R₃ and R₅, or R₃ and R₆, or R₄ and R₅, or R₄ and R₆, together with the carbon atom to which each is attached, join to form a 5- to 6-membered carbocyclic or heterocyclic ring;

R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein;

with a further proviso that A₁, A₂, and A₃ are not all —O—.

Further subsets within the eighteenth subclass of the compounds of Formula (Ia) are those wherein:

A₂ is —O—;

A₂ is —S—;

A₂ is —CR₇R₈—;

A₂ is —NR₉—;

A₂ is —CR₇R₅-A₃-; and

A₂ is -A₃-CR₇R₈—.

A nineteenth subclass of the compounds of Formula (Ia) is that wherein:

A₁ is —CR₇R₈—;

A₂ is —CR₇R₈—, —CR₇R₈-A₃-, or -A₃-CR₇R₈—;

A₃ is —O—, —S—, —NR₉—, or —CR₇R₈—;

Q₁ is —NR₉R₁₀;

Q₂ is O;

R₁ is H;

R₂ is —H, or —C₁-C₄ alkyl;

R₃ and R₅, or R₃ and R₆, or R₄ and R₅, or R₄ and R₆, together with the carbon atom to which each is attached, join to form a 5- to 6-membered carbocyclic or heterocyclic ring;

R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;

each R₉ and R₁₀ are, independently, —H or —C₁-C₄ alkyl;

m is an integer ranging from 1 to 2;

with the proviso that A₁ and A₂ are not —C(OH)₂—;

with the added proviso that when A₁ and A₂ are simultaneously —CH₂—, Q₁ is —NH₂, R₁ is H, R₂ is a —C₂-C₄ linear alkyl or a —C₄ branched alkyl, and Q₂, A₃, n, m, R₃, R₄, R₅, R₆, R₇, R₈, R₉, and R₁₀ are as described herein; and

with a further proviso that A₁, A₂, and A₃ are not all —O—.

Further subsets within the nineteenth subclass of the compounds of Formula (Ia) are those wherein:

A₃ is —O—;

A₃ is —S—;

A₃ is —CR₇R₈—; or

A₃ is —NR₉—.

Exemplary pyrrolidine compounds of Formula (Ia) are:

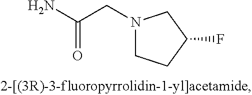

or a pharmaceutically acceptable salt thereof;

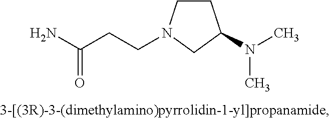

or a pharmaceutically acceptable salt thereof; and
and

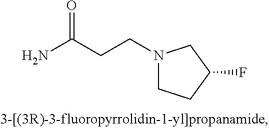

or a pharmaceutically acceptable salt thereof.

An exemplary piperidine compound of Formula (Ia) is:

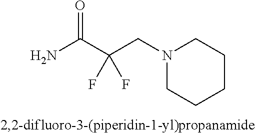

or a pharmaceutically acceptable salt thereof.

Other exemplary compounds of Formula (Ia) are shown below:

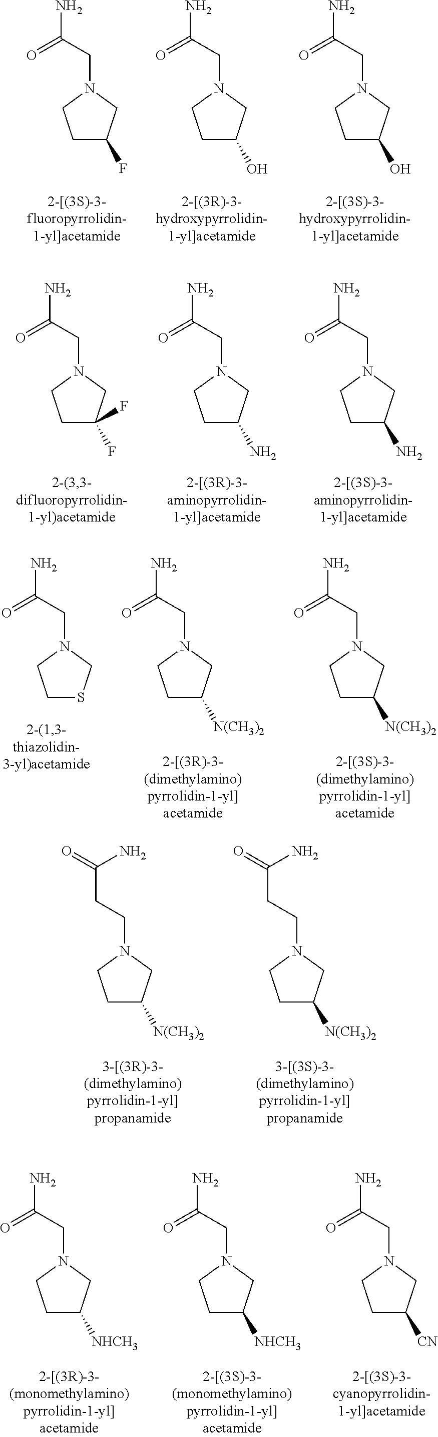

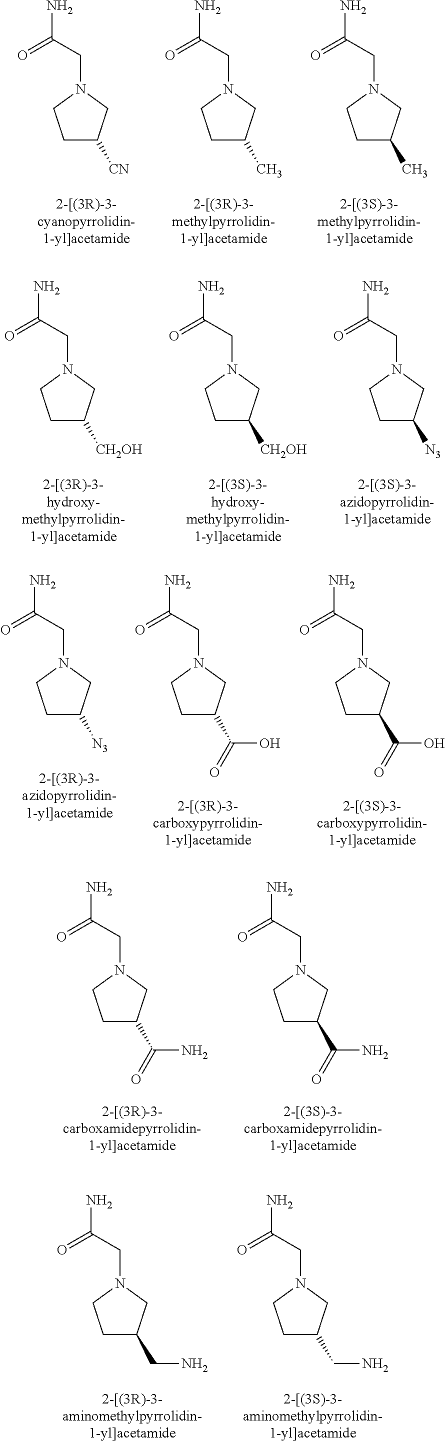

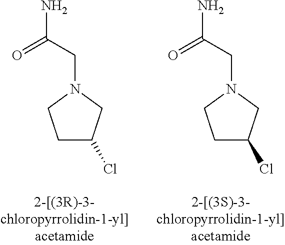

Other illustrative compounds of Formula (Ia) are shown below:

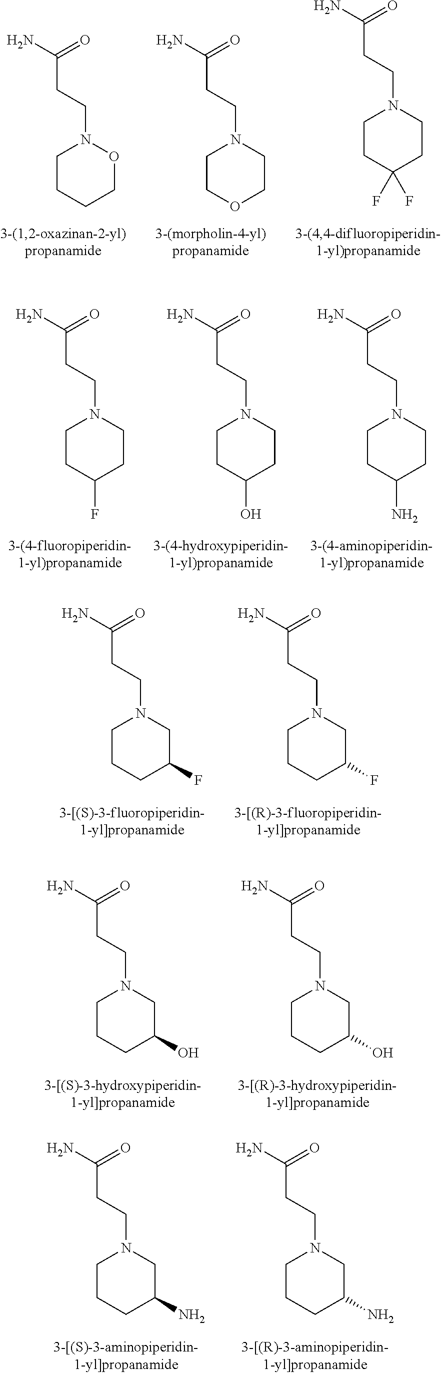

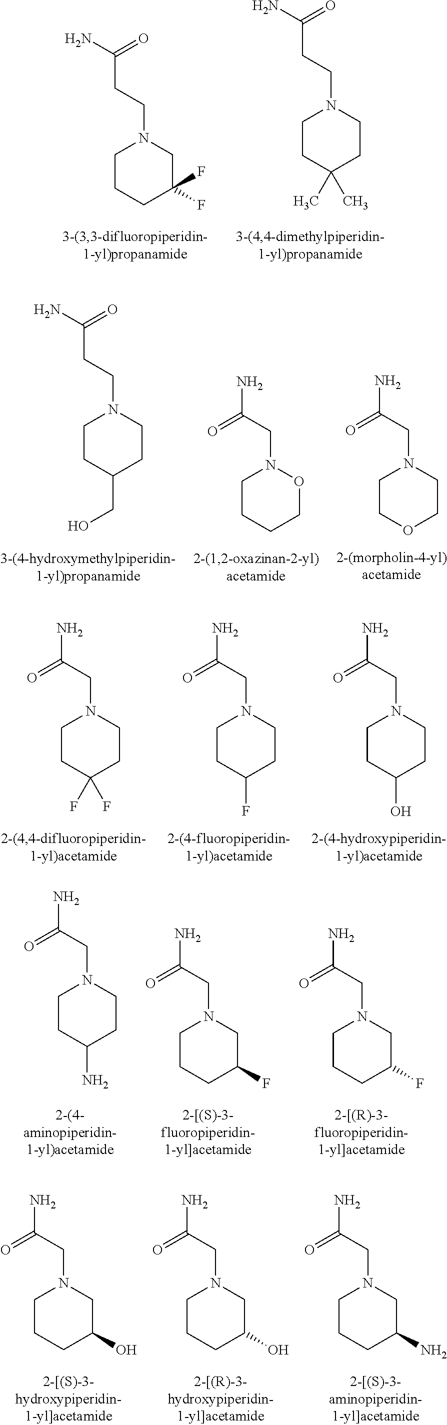

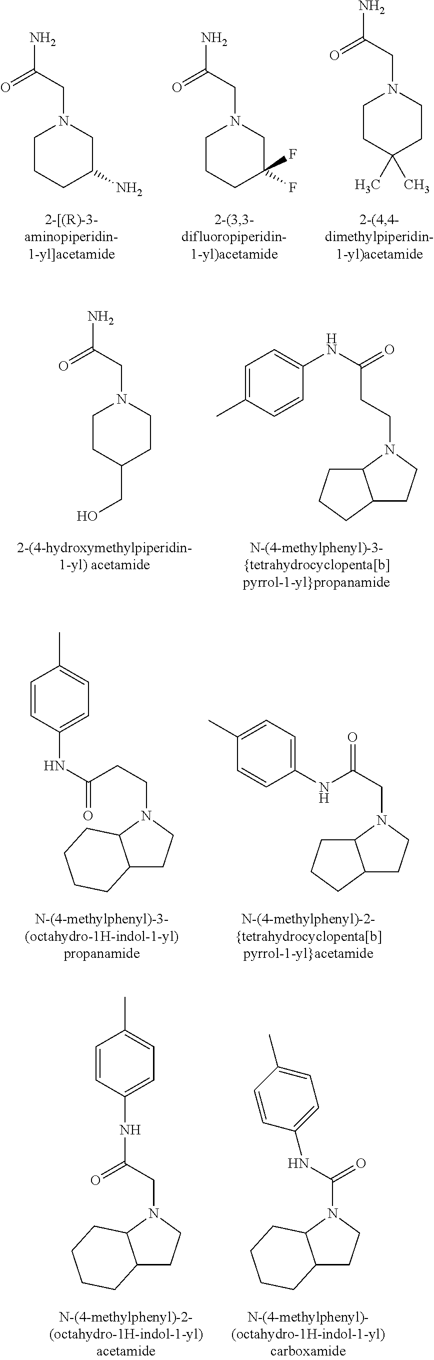

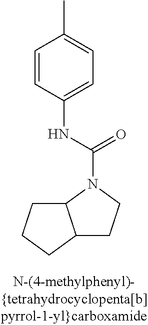

Other compounds of Formula (Ia) are (R)-2-ethyl-2-(pyrrolidin-1-yl)acetamide, (S)-2-ethyl-2-(pyrrolidin-1-yl)acetamide, (R)-2-ethyl-2-(piperidin-1-yl)acetamide, (S)-2-ethyl-2-(piperidin-1-yl)acetamide, (R)-2-(4-methylphenyl)-2-(piperidin-1-yl)acetamide, and (S)-2-(4-methylphenyl)-2-(piperidin-1-yl)acetamide.

The present invention also provides compositions comprising a pharmaceutically acceptable carrier and an effective amount of a compound of Formula (Ia) or a pharmaceutically acceptable salt thereof.

The invention further provides methods for treating or preventing pain by administering to a patient in need of such treatment or prevention an effective amount of a compound of Formula (Ia), or a pharmaceutically acceptable salt thereof.

The invention further provides methods for treating or preventing neuropathic pain by administering to a patient in need of such treatment or prevention an effective amount of a compound of Formula (Ia), or a pharmaceutically acceptable salt thereof.

### Pyrrolidine and Piperidine Compounds of Formula (Ib)

The present invention also provides methods for treating or preventing pain, by administering to a patient in need of such treatment or prevention an effective amount of a compound of Formula (Ib),

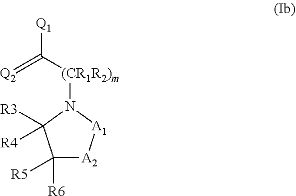

including enantiomers, diastereomers, isomers, prodrugs and pharmaceutically acceptable salts thereof, wherein:

Q₁, Q₂, n, m, A₁, A₂, A₃, R₁, R₂, R₃-R₈, R₉, and R₁₀ are defined above for the compounds of formula (Ib).

A first subclass of the compounds of Formula (Ib) is that wherein:

A₁ is —CR₇R₈—;

A₂ is —CR₇R₈—, —CR₇R₈-A₃-, or -A₃-CR₇R₈—;

A₃ is —CR₇R₃—;

R₁ and R₂ are, independently, —H, —OH, halogen, —CN, —NO₂, —SH, —N₃, —C₁-C₈ alkyl, —C₂-C₆ alkenyl, —C₂-C₆ alkynyl, —C₃-C₆ cycloalkyl, —C₆-C₁₀ aryl, —C₇-C₁₀ arylalkyl, 3 to 7-membered heterocycle, —OR₉, —NHR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)NR₉R₁₀, —C(O)OR₉, —SR₉, —SOR₉, —S(O)₂R₉, —NHC(O)R₉, —NHSR₉, —NHSOR₉, —NHS(O)₂R₉, —OS(O)₂O″, —OS(O)₂R₉, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₁ and R₂, together with the carbon atom to which each is attached, join to form a 3- to 7-membered carbocyclic or heterocyclic ring;

R₃, R₄, R₅, R₆, R₇, and R₈ are, independently, —H, —OH, halogen, —CN, —NO₂, —SH, —N₃, —C₁-C₆ alkyl, —C₂-C₆ alkenyl, —C₂-C₆ alkynyl, —C₃-C₁₀ cycloalkyl, —C₆-C₁₀ aryl, —C₇-C₁₀ arylalkyl, 3 to 7-membered heterocycle, —OR₉, —NHR₉, —NR₉R₁₀, —O(CH₂)ₙOR₉, —C(O)R₉, —OC(O)R₉, —C(O)(CH₂)R₉, —OC(O)OR₉, —OC(O)NHR₉, —OC(O)NR₉R₁₀, —C(O)NR₉R₁₀, —C(O)OR₉, —SR₉, —SOR₉, —S(O)₂R₉, —NHC(O)R₉, —NHSR₉, —NHSOR₉, —NHS(O)₂R₉, —OS(O)₂O⁻, —OS(O)₂R₉, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, NR₉C(NH)NR₉R₁₀, —NHC(NCN)NHR₉, —NR₉C(NCN)NR₉R₁₀ or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;

R₉ is, independently, —H, —C₁-C₈ alkyl, —C₃-C₁₂ cycloalkyl, —C₆-C₁₂ aryl, —C₇-C₁₄ arylalkyl, 3 to 7-membered heterocycle, —C₂-C₈ alkenyl, or —C₂-C₈ alkynyl;

R₁₀ is, independently, —H, alkyl, —C₃-C₁₂ cycloalkyl, —C₆-C₁₂ aryl, —C₇-C₁₄ arylalkyl, 3 to 7-membered heterocycle, —C₂-C₈ alkenyl, or —C₂-C₈ alkynyl;

each n is, independently, an integer ranging from 1 to 3;

m is an integer ranging from 1 to 3; and

with the proviso that A₁ and A₂ are not —C(OH)₂—.

A second subclass of the compounds of Formula (Ib) is that wherein:

A₁ is —CR₂R₈—;

A₂ is —CR₇R₈—, —CR₇R₈-A₃-, or -A₃-CR₇R₈—;

A₃ is —CR₇R₈—;

Q₁ is —OR₇ or —NR₉R₁₀;

Q₂ is O or NR₉;

R₁ and R₂ are, independently, —H, —OH, —F, —Cl, —CN, —C₁-C₈ alkyl, —C₂-C₆ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —SR₉, —SOR₉, —S(O)₂R₉, —NHC(O)R₉, —NHS(O)₂R₉, —NHC(NH)NHR₉, —NR₉C(NH)NHR₉, —NHC(NH)NR₉R₁₀, —NR₉C(NH)NR₉R₁₀; —NHC(NCN)NHR₉, —NR₉C(NCN)NR₉R₁₀, or R₁ and R₂ together with the carbon atom to which each is attached, join to form a 3- to 7-membered carbocyclic or heterocyclic ring;

R₃, R₄, R₅, R₆, R₇, and R₈ are, independently, —H, —OH, —F, —Cl, —CN, —NO₂, —SH, —N₃, —C₁-C₆ alkyl, —C₂-C₆ alkenyl, —C₂-C₆ alkynyl, —OR₉, —NHR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;

R₉ is, independently, —H, —C₁-C₆ alkyl, —C₃-C₇ cycloalkyl, —C₆-C₁₀ aryl, —C₇-C₁₀ arylalkyl, 3 to 7-membered heterocycle, —C₂-C₈ alkenyl, or —C₂-C₈ alkynyl;

R₁₀ is, independently, —H, —C₁-C₆ alkyl, —C₃-C₇ cycloalkyl, —C₆-C₁₀ aryl, —C₇-C₁₀ arylalkyl, 3 to 7-membered heterocycle, —C₂-C₈ alkenyl, or —C₂-C₈ alkynyl;

m is an integer ranging from 1 to 2; and

with the proviso that A₁ and A₂ are not —C(OH)₂—.

A third subclass of the compounds of Formula (Ib) is that wherein:

A₁ is —CR₇R₈—;

A₂ is —CR₇R₈—, —CR₇R₈-A₃-, or -A₃-CR₇R₈—;

A₃ is —CR₇R₈—;

Q₁ is —OR₇, or —NR₉R₁₀;

Q₂ is O;

R₁ is —H or —F;

R₂ is —H, —F, —C₁-C₄ alkyl, —C₂-C₆ alkenyl, —OR₉, or —NR₉R₁₀;

R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 6-membered carbocyclic or heterocyclic ring;

R₉ and R₁₀ are, independently, —H, —C₁-C₄alkyl, —C₃-C₆ cycloalkyl, -phenyl, -benzyl, —C₂-C₄ alkenyl;

m is an integer ranging from 1 to 2; and

with the proviso that A₁ and A₂ are not —C(OH)₂—.

A fourth subclass of the compounds of Formula (Ib) is that wherein:

A₁ is —CR₇R₈—;

A₂ is —CR₇R₈—, —CR₇R₈-A₃-, or -A₃-CR₇R₈—;

A₃ is —CR₇R₈—;

Q₁ is —NR₉R₁₀;

Q₂ is O;

R₁ is —H;

R₂ is —H, —C₁-C₄ alkyl, —C₂-C₄ alkenyl;

R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉, or R₃ and R₅, or R₃ and R₆, or R₄ and R₅, or R₄ and R₆, together with the carbon atom to which each is attached, join to form a 5- to 6-membered carbocyclic or heterocyclic ring;

R₉ and R¹⁰ are, independently, —H, —C₁-C₄ alkyl, —C₃-C₆ cycloalkyl, phenyl, benzyl, —C₂-C₄ alkenyl;

m is an integer ranging from 1 to 2; and

with the proviso that A₁ and A₂ are not —C(OH)₂—.

A fifth subclass of the compounds of Formula (Ib) is that wherein:

A₁ is —CR₇R₈—;

A₂ is —CR₇R₈—, —CR₇R₈-A₃-, or -A₃-CR₇R₈—;

A₃ is —CR₇R₈—;

Q₁ is —NR₉R₁₀;

Q₂ is O;

R₁ is —H;

R₂ is —H or —C₁-C₄ alkyl;

R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉;

R₉ and R₁₀ are, independently, —H, —C₁-C₄ alkyl, -phenyl, -benzyl, or —C₂-C₄ alkenyl;

m is an integer ranging from 1 to 2; and

with the proviso that A₁ and A₂ are not —C(OH)₂—.

A sixth subclass of the compounds of Formula (Ib) is that wherein:

A₁ is —CR₇R₈—;

A₂ is —CR₇R₈—, —CR₇R₈-A₃-, or -A₃-CR₇R₈—;

A₃ is —CR₇R₈—;

Q₁ is —NR₉R₁₀;

Q₂ is O;

R₁ is —H;

R₂ is —H, —C₁-C₄ alkyl, or —C₂-C₄ alkenyl;

R₃ and R₅, or R₃ and R₆, or R₄ and R₅, or R₄ and R₆, together with the carbon atom to which each is attached, join to form a 5- to 6-membered carbocyclic or heterocyclic ring;

R₉ and R₁₀ are, independently, —H, —C₁-C₄ alkyl, -phenyl, -benzyl, or —C₂-C₄ alkenyl;

m is an integer ranging from 1 to 2; and

with the proviso that A₁ and A₂ are not —C(OH)₂—.

A seventh subclass of the compounds of Formula (Ib) is that wherein:

A₁ is —CR₇R₈—;

A₂ is —CR₇R₈—;

Q₁ is —NR₉R₁₀;

Q₂ is O;

R₁ is —H;

R₂ is —H, —C₁-C₄ alkyl, or —C₂-C₄ alkenyl;

R₃ and R₅, or R₃ and R₆, or R₄ and R₅, or R₄ and R₁₃, together with the carbon atom to which each is attached, join to form a 5- to 6-membered carbocyclic or heterocyclic ring;

R₉ and R¹⁰ are, independently, —H, —C₁-C₄ alkyl, -phenyl, -benzyl, or —C₂-C₄ alkenyl;

m is an integer ranging from 1 to 2; and

with the proviso that A₁ and A₂ are not —C(OH)₂—.

An eighth subclass of the compounds of Formula (Ib) is that wherein:

A₁ is —CR₇R₈—;

A₂ is —CR₇R₈-A₃- or -A₃-CR₇R₈—;

A₃ is —CR₇R₈—;

Q₁ is —NR₉R₁₀;

Q₂ is O;

R₁ is —H;

R₂ is —H, —C₁-C₄ alkyl, or —C₂-C₄ alkenyl;

R₃ and R₅, or R₃ and R₆, or R₄ and R₅, or R₄ and R₆, together with the carbon atom to which each is attached, join to form a 5- to 6-membered carbocyclic or heterocyclic ring;

R₉ and R₁₀ are, independently, —H, —C₁-C₄ alkyl, -phenyl, -benzyl, or —C₂-C₄ alkenyl;

m is an integer ranging from 1 to 2; and

with the proviso that A₁ and A₂ are not —C(OH)₂—.

A ninth subclass of the compounds of Formula (Ib) is that wherein:

A₁ is —CR₇R₈—;

A₂ is —CR₇R₈—;

Q₁ is —NH₂;

Q₂ is O;

R₁ is —H;

R₂ is —H or —C₁-C₄ alkyl;

R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉;

R₉ and R₁₀ are, independently, —H, —C₁-C₄ alkyl, -phenyl, -benzyl, or —C₂-C₄ alkenyl;

m is an integer ranging from 1 to 2; and

with the proviso that A₁ and A₂ are not —C(OH)₂—.

A tenth subclass of the compounds of Formula (Ib) is that wherein:

A₁ is —CR₇R₈—;

A₂ is —CR₇R₈-A₃- or -A₃-CR₇R₈—;

A₃ is —CR₇R₈—;

Q₁ is —NH₂;

Q₂ is O;

R₁ is —H;

R₂ is —H or —C₁-C₄ alkyl;

R₃, R₄, R₅, R₆, R₇, and R₈ are, independently, —H, —OH, —F, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉;

R₉ and R₁₀ are, independently, —H, —C₁-C₄ alkyl, -phenyl, -benzyl, or —C₂-C₄ alkenyl;

m is an integer ranging from 1 to 2; and

with the proviso that A₁ and A₂ are not —C(OH)₂—.

An eleventh subclass of the compounds of Formula (Ib) is that wherein:

A₁ is —CR₇R₈—;

A₂ is —CR₇R₈—;

Q₁ is —NH₂;

Q₂ is O;

R₁ is —H;

R₂ is —H, —C₁-C₄ alkyl, or —C₂-C₄ alkenyl;

R₃ and R₅, or R₃ and R₆, or R₄ and R₅, or R₄ and R₆, together with the carbon atom to which each is attached, join to form a 5- to 6-membered carbocyclic or heterocyclic ring;

m is an integer ranging from 1 to 2; and

with the proviso that A₁ and A₂ are not —C(OH)₂—.

A twelfth subclass of the compounds of Formula (Ib) is that wherein:

A₁ is —CR₇R₈—;

A₂ is —CR₇R₈-A₃- or -A₃-CR₇R₈—;

A₃ is —CR₇R₈—;

Q₁ is —NH₂;

Q₂ is O;

R₁ is —H;

R₂ is —H, —C₁-C₄ alkyl, or —C₂-C₄ alkenyl;

R₃ and R₅, or R₃ and R₆, or R₄ and R₅, or R₄ and R₆, together with the carbon atom to which each is attached, join to form a 5- to 6-membered carbocyclic or heterocyclic ring;

m is an integer ranging from 1 to 2; and

with the proviso that A₁ and A₂ are not —C(OH)₂—.

A thirteenth subclass of the compounds of Formula (Ib) is that wherein:

A₁ is —CR₇R₈—;

A₂ is —CR₇R₈—;

Q₁ is —NH₂;

Q₂ is O;

R₁ is —H;

R₂ is —H or —C₁-C₄ alkyl;

R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, or —NHS(O)₂R₉;

R₉ and R₁₀ are, independently, —H, —C₁-C₄ alkyl, phenyl, benzyl, or —C₂-C₄ alkenyl;

m is an integer ranging from 1 to 2; and

with the proviso that A₁ and A₂ are not —C(OH)₂—.

A fourteenth subclass of the compounds of Formula (Ib) is that wherein:

A₁ is —CR₇R₈—;

A₂ is —CR₇R₈-A₃- or -A₃-CR₇R₈—;

A₃ is —CR₇R₃—;

Q₁ is —NH₂;

Q₂ is O;

R₁ is —H;

R₂ is —H or —C₁-C₄ alkyl;

R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, or —NHS(O)₂R₉;

R₉ and R₁₀ are, independently, —H, —C₁-C₄ alkyl, phenyl, benzyl, —C₂-C₄ alkenyl;

m is an integer ranging from 1 to 2; and

with the proviso that A₁ and A₂ are not —C(OH)₂—.

An illustrative pyrrolidine compound of Formula (Ib) is

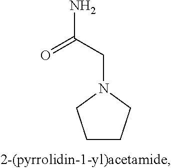

or a pharmaceutically acceptable salt thereof.

An illustrative piperidine compound of Formula (Ib) is

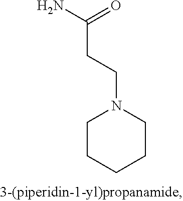

or a pharmaceutically acceptable salt thereof.

Other illustrative compounds of Formula (Ib) are shown below:

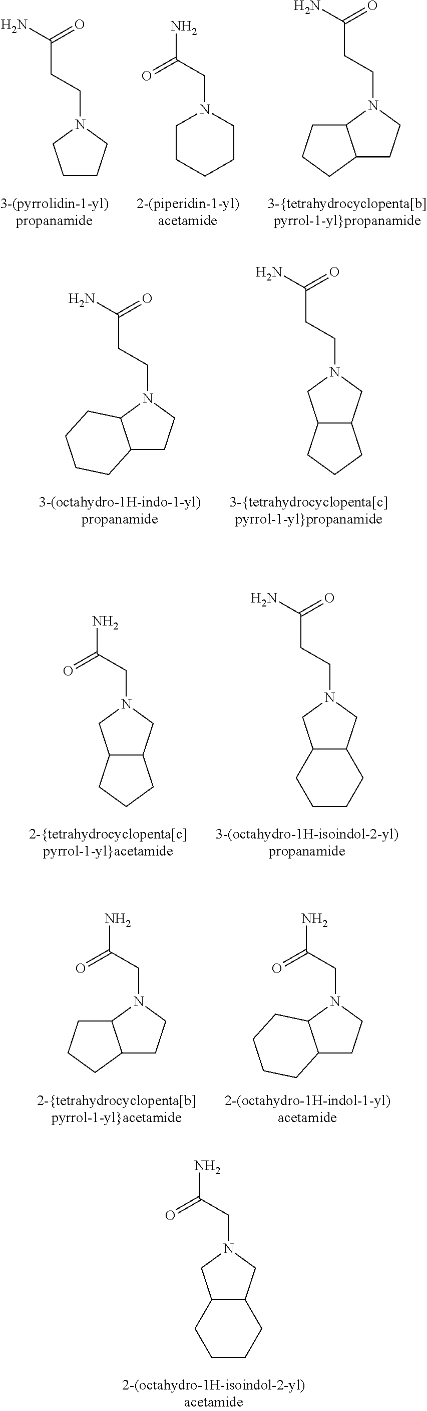

### Methods for Making the Pyrrolidine and Piperidine Compounds

The invention further provides methods useful for making pyrrolidine and piperidine compounds of the invention, and derivatives thereof.

The compounds of the invention can be obtained via standard, well-known synthetic methodology, see e.g. March, J. Advanced Organic Chemistry; Reactions Mechanisms, and Structure, 4^{th} ed., 1992. Illustrative methods are described below. Starting materials useful for preparing the compounds of the invention and their intermediates are also commercially available or can be prepared from commercially available materials using known synthetic methods and reagents.

An example of a synthetic pathway useful for making the compounds of the invention is set forth below and generalized in Scheme 1.

The compounds of Formula (Ia) or (Ib) can be obtained via conventional organic synthesis as described below.

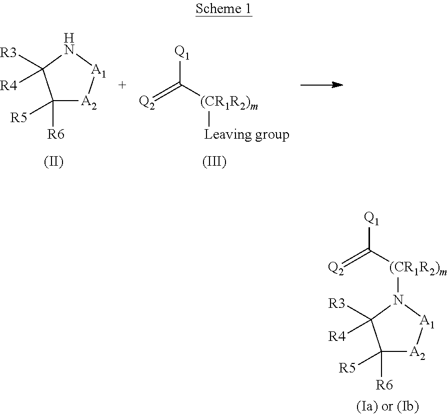

For example, a commercially available or synthetically prepared compound of Formula (II) is subjected to an alkylation reaction with a compound of Formula (III) optionally under basic conditions.

Making the Compounds of Formula (Ia) and (Ib) from the Compounds of Formula (II) and (III) VIA Base Mediated Coupling

In one particular embodiment, the invention provides methods for making compounds of Formula (Ia) or (Ib)

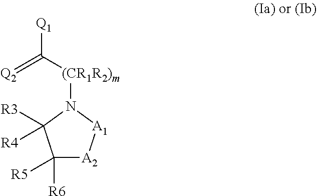

by contacting a compound of Formula (II)

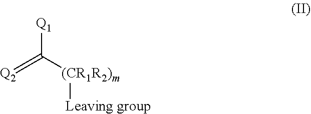

with a compound of Formula (III)

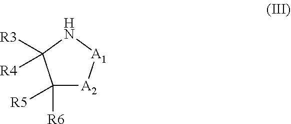

in the presence of an organic solvent such as acetonitrile and optionally a base such as potassium carbonate or sodium hydride, for a time and at a temperature sufficient to make the compound of Formula (Ia) or (Ib). The leaving group from compound (III) is typically a halogen such as bromine or iodine, a C₁-C₆ alkylsulfonate such as methyl triflate, a C₆-C₁₂ arylsulfonate such as tosylate, or a C₁-C₆ alkylsulfide such as thiomethyl.

The formation of a compound of Formula (Ia) or (Ib) can be monitored using conventional analytical techniques, including, but not limited to, thin-layer chromatography (“TLC”), high-performance liquid chromatography (“HPLC”), gas chromatography (“GC”), and nuclear magnetic resonance spectroscopy (“NMR”) such as ¹H or ¹³C NMR.

The concentration of the compound of Formula (III) or of compound of formula (II) in the reaction mixture typically ranges from 0.01 moles to 3 M. In one embodiment, the concentration of the compound of Formula (III) or of compound of Formula (II) in the reaction mixture ranges from 0.05 to 1 M. In another embodiment, the concentration of the compound of Formula (III) in the reaction mixture ranges from 0.1 to 0.5 M of the reaction mixture.

The amount of compound of Formula (II) in the reaction mixture is typically present in at least about a 1.5-fold molar excess to about a 10-fold molar excess relative to the amount of the compound of Formula (III). In one embodiment, the amount of compound of Formula (II) in the reaction mixture is at least about a 2-fold molar excess to about a 10-fold molar excess relative to the amount of the compound of Formula (III). In another embodiment, the amount of compound of Formula (II) in the reaction mixture is at least about a 3-fold molar excess to about a 10-fold molar excess relative to the amount of the compound of Formula (III).

The amount of base in the reaction mixture typically ranges from about 0.0001 to about 5 molar equivalents of the compound of Formula (III). In another embodiment, the amount of base in the reaction mixture ranges from about 0.001 to about 3 molar equivalents of the compound of Formula (III). In another embodiment, the amount of base in the reaction mixture ranges from about 0.01 to about 1 molar equivalents of the compound of Formula (III).

Suitable bases for use in the methods of the invention include, but are not limited to, potassium carbonate, potassium hydride, sodium hydride, potassium t-butoxide, and mixtures thereof. In one embodiment, the base is potassium carbonate or sodium hydride.

The reaction mixture further includes an organic solvent. Suitable organic solvents include, but are not limited to, alcohols, such as methanol, ethanol, isopropanol, and tert-butanol; and ethers, such as diethyl ether, diisopropyl ether, THF, and dioxane. In one embodiment, the solvent is methanol or ethanol.

In one embodiment, the reaction mixture is substantially anhydrous.

The amount of organic solvent in the reaction mixture is typically present at an amount of at least about 10 molar equivalents of the compound of Formula (II). In one embodiment, the organic solvent is present in the reaction mixture in an amount that is at least about 20 molar equivalents of the compound of Formula (II). In another embodiment, the organic solvent is present in the reaction mixture in an amount that is at least about 30 molar equivalents of the compound of Formula (II). In another embodiment, the organic solvent is present in the reaction mixture in an amount that is at least about 40 molar equivalents of the compound of Formula (II).

In one embodiment, the organic solvent is present in the reaction mixture in an amount that ranges from about a 10 molar equivalents to about 1,000 molar equivalents of the compound of Formula (II).

In another embodiment, the organic solvent is present in the reaction mixture in an amount that ranges from about a 20 molar equivalents to about 1,000 molar equivalents of the compound of Formula (II).

In another embodiment, the organic solvent is present in the reaction mixture in an amount that ranges from about a 30 molar equivalents to about 1,000 molar equivalents of the compound of Formula (II).

In another embodiment, the organic solvent is present in the reaction mixture in an amount that ranges from about a 40 molar equivalents to about 1,000 molar equivalents of the compound of Formula (II).

Typically, the reaction proceeds for a time ranging from about 5 minutes to about 24 hours. In one embodiment, the reaction proceeds for a time ranging from about 10 minutes to about 10 hours. In another embodiment, the reaction proceeds for a time ranging from about 30 minutes to about 4 hours.

Typically, the reaction temperature ranges from about 25° C. to about 100° C. In one embodiment, the reaction temperature ranges from about 25° C. to about 40° C. In another embodiment, the reaction temperature is at about room temperature.

Typically, the overall yield of the isolated and purified compound of Formula (Ia) or (Ib) is greater than about 50 percent based on the amount of the compound of Formula (III). In one embodiment, the overall yield of the isolated and purified compound of Formula (Ia) or (Ib) is greater than about 40 percent based on the amount of the compound of Formula (III). In another embodiment, the overall yield of the isolated and purified compound of Formula (Ia) or (Ib) is greater than about 70 percent based on the amount of the compound of Formula (Ia) or (Ib), or on the amount of the compound of Formula (III).

### Therapeutic/Prophylactic Administration and Compositions

Because of their activity, the compounds of the invention are advantageously useful in veterinary and human medicine. For example, the compounds of Formulas (Ia) and (Ib) described herein are useful for the treatment or prevention of pain.

The invention provides methods of treatment and prophylaxis by administration to a patient of an effective amount of a compound of the invention. The patient is an animal, including, but not limited to, a human, mammal, e.g., cow, horse, sheep, pig, cat, dog, mouse, rat, rabbit, mouse or guinea pig, or other animal such as a chicken, turkey, or quail.

The present compositions, which comprise an effective amount of a compound of the invention, can be administered by any convenient route, for example by infusion or bolus injection, or by absorption through epithelial or mucocutaneous linings (e.g., oral mucosa, rectal, intestinal mucosa, etc.), and can be administered together with another biologically active agent. Administration can be systemic or local. Various delivery systems are known, e.g., encapsulation in liposomes, microparticles, microcapsules, capsules, etc., and can be used to administer a compound. In certain embodiments, more than one compound is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically to the ears, nose, eyes, or skin. The preferred mode of administration is left to the discretion of the practitioner.

In specific embodiments, it may be desirable to administer one or more compounds locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an injury. In another embodiment, administration can be by direct injection at the site (or former site) of an infection, tissue or organ transplant, or autoimmune response.

In certain embodiments, it may be desirable to introduce one or more compounds into the central nervous system by any suitable route, including intraventricular and intrathecal injection. Intraventricular injection may be facilitated by an intraventricular catheter, for example, attached to a reservoir, such as an Ommaya reservoir.

Pulmonary administration can also be employed, e.g., by use of an inhaler or nebulizer, formulating with an aerosolizing agent, or via perfusion in a fluorocarbon or synthetic pulmonary surfactant. In certain embodiments, the compounds can be formulated as a suppository, with traditional binders and carriers such as triglycerides.

In another embodiment, the compounds can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compounds can be delivered in a controlled-release system. In one embodiment, a pump may be used (see Langer, supra; Sefton, CRC Crit. Ref. Biomed. Eng. 9:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled-release system can be placed in proximity of the target of the compounds of the invention, e.g., the brain, thus requiring only a fraction of the systemic dose (see, e.g., Goodson, in Medical Applications of Controlled Release, supra, vol. 2, pp. 115-138 (1984)). Other controlled-release systems discussed in the review by Langer (Science 249:1527-1533 (1990)) may be used.

The present compositions may include an effective amount of a compound of the invention and a pharmaceutically acceptable carrier.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in “Remington's Pharmaceutical Sciences” by E. W. Martin.

The compounds included in the present compositions that include an amino moiety may form pharmaceutically acceptable salts with various amino acids, in addition to the acids mentioned above. Compounds, included in the present compositions, that are acidic in nature are capable of forming base salts with various pharmacologically or cosmetically acceptable cations. Examples of such salts include alkali metal or alkaline earth metal salts and, particularly, calcium, magnesium, sodium, lithium, zinc, potassium, and iron salts.

In another embodiment, the compounds of the invention are formulated in accordance with routine procedures as a pharmaceutical composition adapted for intravenous administration to human beings. Typically, compounds for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate may also be used. Oral compositions can include standard carriers such as mannitol, lactose, starch, magnesium stearate, sodium saccharine, cellulose, or magnesium carbonate. Such carriers can be of pharmaceutical grade.

The amount of the compound that will be effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable effective dosage ranges for intravenous administration are generally about 0.01 to about 5 g, preferably about 0.01 to about 1 g of the compound per kilogram body weight. In specific embodiments, the i.v. dose is about 0.005 to about 0.5 g/kg, about 0.01 to about 0.3 g/kg, about 0.025 to about 0.25 g/kg, about 0.04 to about 0.20 g/kg, or about 0.05 to about 0.20 g/kg (or the equivalent doses expressed per square meter of body surface area). Alternatively, a suitable dose range for i.v. administration may be obtained using doses of about 1 to about 2000 mg, without adjustment for a patient's body weight or body surface area. Suitable dosage ranges for intranasal administration are generally about 0.01 pg/kg body weight to 10 mg/kg body weight. Suppositories generally contain 0.5% to 20% by weight of one or more compounds of the invention alone or in combination with another therapeutic agent. Oral compositions can contain about 10% to about 95% by weight of one or more compounds of the invention alone or in combination with another therapeutic agent. In specific embodiments of the invention, suitable dose ranges for oral administration are generally about 0.1 to about 200 mg, preferably about 0.5 to about 100 mg, and more preferably about 1 to about 50 mg of a compound of the invention per kilogram body weight or their equivalent doses expressed per square meter of body surface area. In specific embodiments the oral dose is about 0.25 to about 75 mg/kg, about 1.0 to about 50 mg/kg, about 2.0 to about 25 mg/kg, about 2.5 to about 15 mg/kg, or about 5.0 to about 20 mg/kg (or the equivalent doses expressed per square meter of body surface area). In another embodiment, a suitable dose range for oral administration, from about 10 to about 4000 mg, without adjustment for a patient's body weight or body surface area. Other effective doses may be extrapolated from dose-response curves derived from in vitro or animal model test systems. Such animal models and systems are well known in the art.

The invention also provides pharmaceutical packs or kits comprising one or more containers containing one or more compounds. Optionally associated with such container(s) can be a notice in the form prescribed by a governmental agency regulating the manufacture, use or sale of pharmaceuticals or biological products, which notice reflects approval by the agency of manufacture, use or sale for human administration. In certain embodiments, e.g., when administered for the treatment or prevention of pain, the kit may also contain one or more analgesic agents useful for treating pain to be administered in combination with a compound of the invention.

### Treatment or Prevention of Pain Further Comprising Administering Other Pain Control Agents

Pain can be treated or prevented by administration of an effective amount of a compound of the invention.

In certain embodiments, the present methods for treating or preventing pain further comprise administering an effective amount of the compounds of the invention and one or more pain control agent, including, but not limited to, gababentin, morphine, oxycodone, fentanyl, pethidine, methadone, propoxyphene, hydromorphone, hydrocodone, codeine, meperidine, gabapentin, pregabalin, lidocaine, ketamine, capsaicin, anticonvulsants such as valproate, oxcarbazepine or carbamazepine, tricyclic antidepressants such as amitriptyline, duloxetine, venlafaxine, and milnacipran.

### Prodrugs

The present invention also provides prodrugs of the compounds of the invention. Prodrugs include derivatives of the compounds of the invention that can hydrolyze, oxidize, or otherwise react under biological conditions (in vitro or in vivo) to provide an active compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, and biohydrolyzable phosphate analogues. In certain embodiments, prodrugs of the compounds of the invention with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh). Biohydrolyzable moieties of a compounds of the invention
1) do not interfere with the biological activity of the compound but can confer upon that compound advantageous properties in vivo, such as uptake, duration of action, or onset of action; or
2) are biologically inactive but are converted in vivo to the biologically active compound.
   Examples of biohydrolyzable esters include, but are not limited to, lower alkyl esters, alkoxyacyloxy esters, alkyl acylamino alkyl esters, and choline esters. Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

### EXAMPLES

The examples below are provided for the purpose of illustrating the invention and should not be construed as limiting.

### Example 1

Compounds of Formula (Ia) and (Ib) are prepared by using the general procedure as shown in Scheme 2 below.

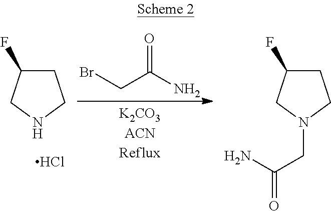

To a mixture of pyrrolidine compound, e.g., (S)-(+)-3-fluoropyrrolidine hydrochloride, (2.0 g, 15.9 mmol) in anhydrous acetonitrile (30 mL) was added potassium carbonate (4.83 g, 35.0 mmol). The mixture was stirred at room temperature for 5 minutes before 2-bromoacetamide (2.08 g, 15.1 mmol) was added. The mixture was stirred at reflux overnight. The warm mixture was filtered. The filtrate was recovered, evaporated under reduced pressure and dried in vacuo, affording the depicted compound.

### Preparation of 2-(1,3-thiazolidin-3-yl)acetamide

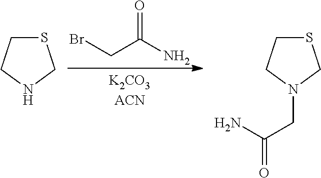

(1.01 g, 21%). ¹H NMR (400 MHz, DMSO-d6) 2.78 (t, J=6.5 Hz, 2H), 2.87 (s, 2H), 3.00 (t, J=6.3 Hz, 2H), 7.16 (s (br), 1H), 7.35 (s (br), 1H), M+ 147.

### Preparation of 2-[(3S)-3-fluoropyrrolidin-1-yl]acetamide

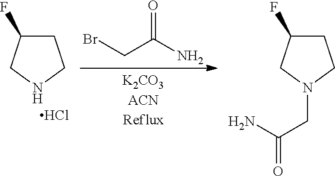

(1.91 g, 87%). ¹H NMR (400 MHz, CD₃OD-d4) 2.02 (m, 1H), 2.15 (m, 1H), 2.56 (m, 1H), 2.80 (m, 0.5H), 2.92 (m, 2.5H), 3.18 (s, 2H), 5.10 (m, 0.5H), 5.24 (m, 0.5H), M+ 147.

### Preparation of 2-[(3R)-3-fluoropyrrolidin-1-yl]acetamide

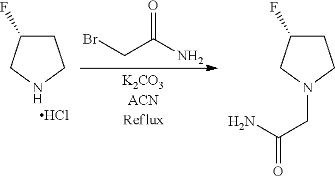

The filtrate was recovered, evaporated under reduced pressure, and dried in vacuo, affording the title compound (1.64 g, 88%). ¹H NMR (400 MHz, CD₃OD-d4) 2.01 (m, 1H), 2.19 (m, 1H), 2.56 (m, 1H), 2.80 (m, 0.5H), 2.92 (m, 2.5H), 3.18 (s, 2H), 5.11 (m, 0.5H), 5.24 (m, 0.5H), M+ 147.

### Preparation of 3-[(3R)-3-fluoropyrrolidin-1-yl]propanamide

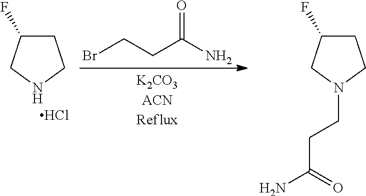

The crude product was purified by flash chromatography (reverse phase (C₁₈ column), 0-10% ACN/5 mM NH₄OH_{(aq)}), affording the title compound, (0.728 g, 40%). ¹H NMR (400 MHz, CD₃OD-d4) 2.00 (m, 1H), 2.19 (m, 1H), 2.44 (m, 3H), 2.65 (m, 0.5H), 2.72 (m, 0.5H), 2.79 (t, J=7.3 Hz, 2H), 2.95 (m, 2H), 5.10 (m, 0.5H), 5.23 (m, 0.5H). M+ 161.

### Preparation of 3-[(3S)-3-fluoropyrrolidin-1-yl]propanamide

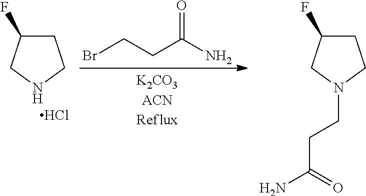

The crude product was purified by flash chromatography (reverse phase (C₁₈ column), 0-10% ACN/5 mM NH₄OH_{(aq)}), affording the title compound, (0.538 g, 30%). ¹H NMR (400 MHz, CD₃OD-d4) 1.99 (m, 1H), 2.19 (m, 1H), 2.43 (m, 3H), 2.62 (m, 0.5H), 2.72 (m, 0.5H), 2.78 (t, J=7.3 Hz, 2H), 2.94 (m, 2H), 5.11 (m, 0.5H), 5.23 (m, 0.5H), M+ 161.

### Preparation of 2-[(3R)-3-(dimethylamino)pyrrolidin-1-yl]acetamide

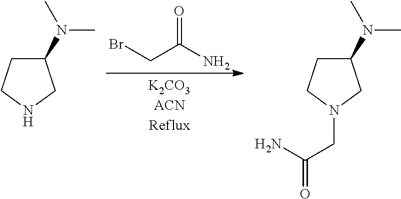

The crude product was purified by flash chromatography (reverse phase (C₁₈ column), 0-30% ACN/5 mM NH₄OH_{(aq)}), affording the title compound, (394 mg, 18%). ¹H NMR (400 MHz, DMSO-d6) 1.58 (m, 1H), 1.83 (m, 1H), 2.08 (s, 6H), 2.31 (t, J=6.7 Hz, 1H), 2.47 (m, 1H), 2.70 (m, 3H), 2.94 (q, J=16.6 Hz, 2H), 7.08 (bs, 2H), M+ 172.

### Preparation of 3-[(3R)-3-(dimethylamino)pyrrolidin-1-yl]propanamide

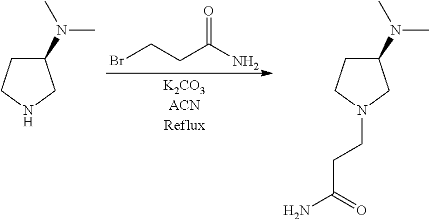

The crude product was purified by flash chromatography (reverse phase (C₁₈ column), 0-10% ACN/5 mM NH₄OH_{(aq)}), affording the title compound (460 mg, 19%). ¹H NMR (400 MHz, DMSO-d6) 1.55 (m, 1H), 1.79 (m, 1H), 2.07 (s, 6H), 2.26 (m, 3H), 2.36 (m, 1H), 2.48 (m, 1H), 2.61 (m, 4H), 6.75 (s (br), 1H), 7.35 (s (br), 1H), M+ 186.

### Preparation of 2-(1,2-oxazinan-2-yl)acetamide

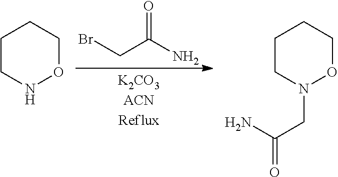

The crude product was purified by flash chromatography (reverse phase (C₁₈ column), 0-10% ACN/5 mM NH₄OH_{(aq)}), affording the title compound as a light-pink solid, 410 mg (55%); (410 mg, 55%); ¹H NMR (400 MHz, DMSO-d6) 1.46 (m, 2H), 1.71 (m, 2H), 2.54 (m, 2H), 3.10 (m, 2H), 3.86 (t, J=5.5 Hz, 2H), 6.97 (s (br), 1H), 7.15 (s (br), 1H), M+ 145.

### 2-[(3S)-3-(dimethylamino)pyrrolidin-1-yl]acetamide

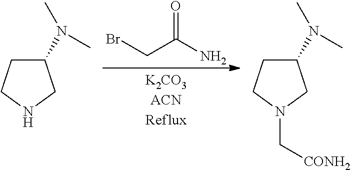

(2.1 g, 56%), ¹H NMR (400 MHz, DMSO-d6) 1.78 (m, 1H), 2.01 (m, 1H), 2.25 (s, 6H), 2.76 (m, 5H), 3.12 (m, 2H).

### 3-[(3S)-3-(dimethylamino)pyrrolidin-1-yl]propanamide

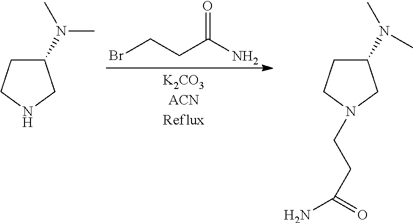

(3.1 g, 78.3%) ¹H NMR (400 MHz, DMSO-d6) 1.79 (m, 1H), 1.55 (m, 1H), (m, 1H), 2.04 (s, 6H), 2.20 (m, 3H), 2.35 (m, 1H). 2.45-2.66 (m, 5H).

### 2-[(3S)-3-hydroxypyrrolidin-1-yl]acetamide

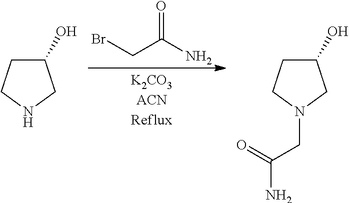

(1.91 g, 87%). ¹H NMR (400 MHz, DMSO-d6): 1.54 (m, 1H), 1.97 (m, 1H), 2.42 (m, 2H), 2.64 (m, 2H), 2.80 (m, 0.5H), 2.92 (m, 2H), 4.12 (bs, 1H), 4.71 (bs, 1H), 7.07 (bs, 1H), 7.20 (bs, 1H).

### 2-(1,2-oxazolidin-2-yl)acetamide

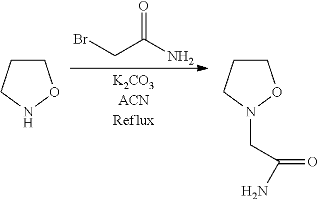

(97 mg, 11%). ¹H NMR (400 MHz, DMSO-d6) 1.29 (m, 1H), 1.62 (m, 1H), 2.50 (m, 1H), 2.76 (m, 1H), 3.02 (m, 1H), 3.62 (t, J=9.4, 11.7 Hz, 1H), 3.97 (m, 1H), 4.40 (s, 1H), 7.14, 7.35 (2bs, 2H), M+ 130.9

### 2-(3,3-difluoropyrrolidin-1-yl)acetamide

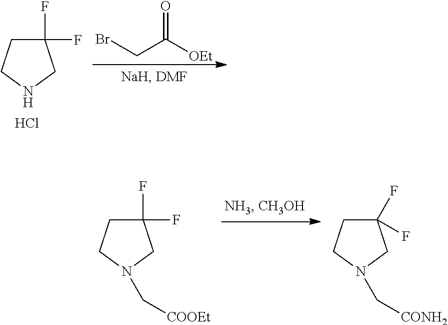

To the suspension of NaH (924 mg, 23.1 mmol) in DMF (40 mL), 3,3′-difluoropyrrolidine (1.57 g, 11.00 mmol) was added at 0° C. After 2 h, ethyl 2-bromoacetate (1.34 mL, 12.10 mmol) was added. The resulting mixture was stirred at r.t. overnight. The mixture was partitioned among water (400 mL) and ethyl acetate The organic layers were combined and washed with water and brine and dried over MgSO₄. The crude ester was dissolved in 20 mL of 7N NH₃ solution in methanol and was stirred over night. Solvent was evaporated and purified by column to provide the title compound.

(1.2 g, 50%), ¹H NMR (400 MHz, DMSO-d6) 2.19-2.30 (m, 2H), 2.78 (t, 2H), 3.00 (m, 4H), 7.09-7.24 (d, 2H, NH₂),

### 2-(pyrrolidin-1-yl)acetamide

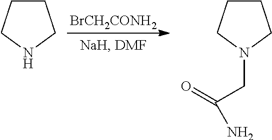

To the mixture of pyrrolidine (2.13 g, 30 mmol) in DMF (60 mL) at 0° C., NaH (1.6 g, 60%, 40.00 mmol) was added. The mixture was stirred vigorously for 2 h. Bromoacetamide (8.28 g, 60.00 mmol) was added. The resulting mixture was stirred at r.t overnight. After being diluted with water (300 mL), the mixture was extracted with AcOEt (200 mL×3). The organic layers were combined and dried over MgSO₄ and evaporated. The crude was product recrystallized from THF/ether to obtain a white crystal solid (13%, 500 mg).

¹H-NMR (CDCl₃, 400 MHz): 1.81 (m, 4H), 2.63 (m, 4H), 3.15 (s, 2H), 6.03, 7.02 (2bs, 2H).

### 3-(piperidin-1-yl)propanamide

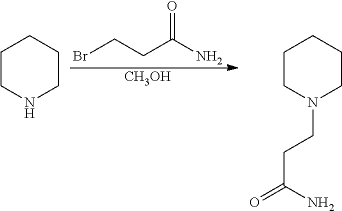

The mixture of piperidine (2.0 mL, 20 mmol) and bromo propionamide (1.52 g, 10 mmol) in CH₃OH (20 mL) was heated at 70° C. over 48 h. The solvent was evaporated. The crude was recrystallized from methanol/diethyl ether to provide a white solid (22.4%, 760 mg). ¹H NMR (400 MHz, DMSO-d6): 1.35 to 1.50 (m, 6H), 2.18 to 2.50 (m, 10H), 6.75, 7.38, (2bs, 2H).

### 3-(pyrrolidin-1-yl)propanamide

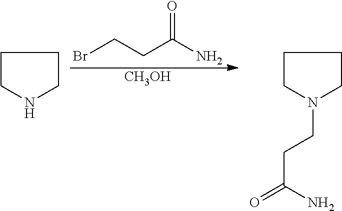

The mixture of pyrrolidine (5.0 mL, 50.50 mmol) and bromo propionamide (2.0 g, 13.1 mmol) in CH₃OH (25 mL) was heated at 80° C. over night. The solvent was evaporated. The crude was recrystallized from methanol/diethyl ether to provide white solid (17.1%, 1.20 g). ¹H NMR (400 MHz, DMSO-d6): 1.91 (m, 4H), 2.60 (m, 2H), 2.99 to 3.40 (m, 6H), 7.62, 7.09 (2bs, 2H).

### 2-cyclopentylacetamide

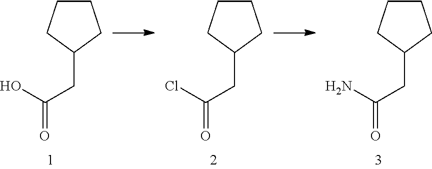

To a solution of cyclopentylacetic acid (1) (5.0 g, 39.01 mmol) in dichloromethane 50 mL at 0° C. was added oxalyl chloride (5.94 g, 46.81 mmol) dropwise, followed by a drop of DMF. Reaction was allowed to stir overnight at room temperature. Solvent was evaporated to provide a crude oil, which was dissolved in acetonitrile 30 ml. Then the crude acid chloride 2 was slowly added to a cooled solution of 30% ammonium hydroxide. Then the reaction was allowed to stir at room temperature. After the reaction was complete (30 min) solvent was evaporated. The product crystallized out, was filtered off, and washed with water and ether to provide a white solid 3 (2.70 g, 54.4%). ¹H NMR (400 MHz, DMSO-d6) 1.09 (m, 2H), 1.44-1.74 (m, 6H), 2.03 (m, 2H), 2.11 (m, 1H), 6.67 (bs, 1H), 7.21 (bs, 1H). M+ 128.2.

### 2-(2-oxo-1,3-thiazolidin-3-yl)acetamide

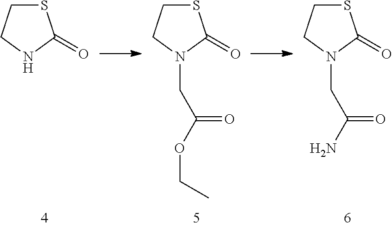

A solution of 2-thiazolidinone (4) (3.0 g, 29.08 mmol) in acetonitrile (50 mL) was added K₂CO₃ (9.63 g, 69.80 mmol) and ethyl bromoacetate ((5.82 g, 34.90 mmol). A catalytic amount of 18-crown-6 was added. The reaction mixture was heated to reflux for 4 hrs and was cooled to room temperature and filtered. The filtrate was evaporated to dryness and was extracted into dichloromethane (2×50 mL) and washed with 1 M-KCl solution. The organic layer was separated and dried over anhydrous Na₂SO₄ and filtered. The filtrate was evaporated to provide the crude product 5, which was used for the next reaction without any further purification.
The crude alkylated compound 5 was dissolved in minimum amount of methanol and 7N—NH₃-MeOH was added and was stirred over night. The amide precipitated out, was filtered off, and was washed with ether to provide (1.5 g, 32.05%) of the amide 6. ¹H NMR (400 MHz, DMSO-d6): 3.30 (m, 2H), 3.62 (m, 2H), 3.80 (s, 2H), 7.11 (bs, 1H), 7.44 (bs, 1H). M+ 161.4.

### Example 2

### Analgesic Effects of the Compounds of the Invention

### Determination of Analgesic Effect in an Experimental Model of Neuropathic Pain

The analgesic effects of 2-(pyrrolidin-1-yl)acetamide, 3-(piperidin-1-yl)propanamide, 3-[(3S)-3-fluoropyrrolidin-1-yl]propanamide, and 3-[(3R)-3-(dimethylamino)pyrrolidin-1-yl]propanamide were determined using the procedures described hereafter. Other methods will be known to the skilled artisan.

Adult, male Sprague-Dawley rats were obtained from Charles River Laboratories (St Constant, QC) and housed under standard conditions at the Institut Armand-Frappier (Laval, QC). Food and water were provided to experimental animals ad libitum, and rats weighed 175-200 grams at the time of assessment.

Compounds were prepared for intrathecal administration by dissolving them in a vehicle of D5W (5% dextrose); total volume of solution administered to rats was 20 μl, and the quantity of representative compounds used was ˜60 mmol.

Neuropathic pain was induced in rats via chronic constriction injury (CCI) of the left sciatic nerve in accordance with the procedure described by Bennett & Xie (Pain, 1988). Briefly, under ketamine/xylazine anaesthesia, the sciatic nerve was exposed by dissection at the level of mid-thigh, and four loose ligatures (USP 4/0, Braun Melsungen, FRG) were implanted around the nerve with due attention not to interrupt the epineural circulation. The incision was closed-up using simple suturing, and the rats allowed to recover.

After two weeks, a stable allodynia to blunt mechanical stimuli was identified in the hind paw ipsilateral to the CCI, manifested as a reduction of 50% withdrawal threshold, and identified using the Von Frey technique, as described by Chaplan et al. (Journal of Neuroscience Methods, 1994). Rats were considered to be fully neuropathic upon displaying a 50% withdrawal threshold of ≦3.5 grams consistently over the course of 72 hours.

Under brief isoflurane analgesia, compounds were administered to neuropathic rats via acute local delivery in the intrathecal space surrounding the lumbar enlargement of the spinal cord.

Thirty minutes following intrathecal administration of representative compounds to neuropathic rats, the 50% withdrawal threshold rose from a mean 2.9±0.1 g to a mean 6.0±0.9 g (significantly higher than that evoked by vehicle, p<0.05, as assessed by repeated-measures ANOVA). Sixty minutes post-administration of compounds, the mean 50% withdrawal threshold was 6.1±0.9 g (p<0.05 compared to vehicle).

It should also be noted that for in vivo medicinal uses, potency is not the only factor to be considered to estimate the suitability of a compound as a pharmaceutical agent. Other factors such as toxicity and bioavailability also determine the suitability of a compound as a pharmaceutical agent. Toxicity and bioavailability can also be tested in any assay system known to the skilled artisan.

### Other Embodiments

The present invention is not to be limited in scope by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which are incorporated herein by reference.

Other embodiments are in the claims.

What is claimed is:

1. A compound having the structure depicted as Formula (Ia) 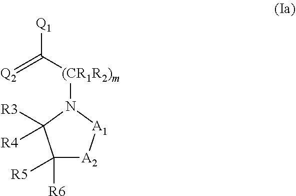
including enantiomers, diastereomers, isomers, prodrugs and pharmaceutically acceptable salts thereof, wherein:
A₁ is —O—, —S—, —SO—, —SO₂—, —CR₇R₈—, —C(O)—, —NH—, —NR₉—, —NOR₉—, —NC(O)R₉—, —NSR₉—, —NSOR₉—, —NS(O)₂R₉—, —NC(S)R₉—, —NC(S)NHR₉—, —NC(S)NR₉R₁₀—, —NC(NH)NHR₉—, —NC(NH)NR₉R₁₀—, —NC(NCN)NHR₉—, or —NC(NCN)NR₉R₁₀—;
A₂ is —O—, —S—, —SO—, —SO₂—, —CR₇R₈, —CR₇R₈-A₃-, -A₃-CR₇R₈—, —NH—, —NR₉—, —NOR₉—, —NC(O)R₉—, —NSR₉—, —NSOR₉—, —NS(O)₂R₉—, —NC(S)R₉—, —NC(S)NHR₉—, —NC(S)NR₉R₁₀—, —NC(NH)NHR₉—, —NC(NH)NR₉R₁₀—, —NC(NCN)NHR₉—, or —NC(NCN)NR₉R₁₀—;
A₃ is —O—, —S—, —SO—, —SO₂—, —CR₇R₈, —NH—, —NR₉—, —NOR₉—, —NC(O)R₉—, —NSR₉—, —NSOR₉—, —NS(O)₂R₉—, —NC(S)R₉—, —NC(S)NHR₉—, —NC(S)NR₉R₁₀—, —NC(NH)NHR₉—, —NC(NH)NR₉R₁₀—, —NC(NCN)NHR₉—, or —NC(NCN)NR₉R₁₀—;
Q₁ is —OR₇, —SR₇ or —NR₉R₁₀;
Q₂ is O, S, NH, or NR₉;
R₁ and R₂ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NHOR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O″, —OC(S)R₉, —OC(S)OR₉, —OC(S)NHR₉, —OC(S)NHNR₉R₁₀, —C(S)OR₉, —C(S)NHR₉, —C(S)NHNR₉R₁₀; —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀ or R₁ and R₂ together with the carbon atom to which each is attached, join to form a 3- to 9-membered carbocyclic or heterocyclic ring;
R₃, R₄, R₅, R₆, R₇, and R₈ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NHOR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀ or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;
each R₉ and R₁₀ is, independently, —H, —C₁-C₈ alkyl, —C₃-C₁₂ cycloalkyl, —C₆-C₁₂ aryl, —C₇-C₁₄ arylalkyl, 3- to 9-membered heterocycle, —C₂-C₈ alkenyl, or —C₂-C₈ alkynyl;
each n is, independently, an integer ranging from 1 to 5;
m is an integer ranging from 0 to 3;
with the proviso that A₁ and A₂ are not —C(OH)₂—;
with the added proviso that when A₁ and A₂ are both —CH₂—, Q₁ is —NH₂, Q₂ is O, R₁ is H, R₂ is a —C₂-C₈ linear alkyl or a —C₄-C₈ branched alkyl, m=1 to 3; and
with a further proviso that A₁, A₂, and A₃ are not all —O—.

2. A compound of claim 1, wherein:
A₁ is —O—, —S—, —SO—, —SO₂—, —CR₇R₈, —NH—, —NR₉—, —NOR₉—, —NC(O)R₉—, —NSR₉—, —NSOR₉, —NS(O)₂R₉, —NC(S)R₉, —NC(S)NHR₉, —NC(S)NR₉R₁₀, —NC(NH)NHR₉, —NC(NH)NR₉R₁₀, —NC(NCN)NHR₉, or —NC(NCN)NR₉R₁₀;
A₂ is —O—, —S—, —SO—, —SO₂—, —CR₇R₈, —CR₇R₈-A₃-, -A₃-CR₇R₈—, —NH—, —NR₉—, —NOR₉—, —NC(O)R₉, —NSR₉, —NSOR₉, —NS(O)₂R₉, —NC(S)R₉, —NC(S)NHR₉, —NC(S)NR₉R₁₀, —NC(NH)NHR₉, —NC(NH)NR₉R₁₀, —NC(NCN)NHR₉, or —NC(NCN)NR₉R₁₀;
A₃ is —O—, —S—, —SO—, —SO₂—, —CR₇R₃, —NH—, —NR₉—, —NOR₉—, —NC(O)R₉, —NSR₉, —NSOR₉, —NS(O)₂R₉, —NC(S)R₉, —NC(S)NHR₉, —NC(S)NR₉R₁₀, —NC(NH)NHR₉, —NC(NH)NR₉R₁₀, —NC(NCN)NHR₉, or —NC(NCN)NR₉R₁₀;
Q₁ is —NR₉R₁₀;
Q₂ is O;
R₁ is —H;
R₂ is —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NHOR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, O—C(S)R₉, O—C(S)OR₉, O—C(S)NHR₉, O—C(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₁ and R₂ together with the carbon atom to which each is attached, join to form a 3- to 9-membered carbocyclic or heterocyclic ring;
R₃, R₄, R₅, R₆, R₇, and R₈ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NHOR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;
R₉ and R₁₀ are, independently, —H, —CH₃, —CH₂CH₃, or —CH(CH₃)₂, -phenyl, or -benzyl; and
each n is, independently, an integer ranging from 1 to 2;
including isomers, prodrugs and pharmaceutically acceptable salts thereof.

3. A compound of claim 1, wherein:
A₁ is —O—, —S—, —SO—, —SO₂—, —CR₇R₈, —NH—, —NR₉—, —NOR₉—, —NC(O)R₉, —NS(O)₂R₉, —NC(NH)NHR₉, —NC(NH)NR₉R₁₀, —NC(NCN)NHR₉, or —NC(NCN)NR₉R₁₀;
A₂ is —O—, —S—, —SO—, —SO₂—, —CR₇R₃, —CR₇R₈-A₃-, -A₃-CR₇R₉—, —NH—, —NR₉—, —NOR₉—, —NC(O)R₉, —NS(O)₂R₉, —NC(S)R₉, —NC(NH)NHR₉, —NC(NH)NR₉R₁₀, —NC(NCN)NHR₉, or —NC(NCN)NR₉R₁₀;
A₃ is —O—, —S—, —SO—, —SO₂—, —CR₇R₈, —NH—, —NR₉—, —NOR₉—, —NC(O)R₉, —NS(O)₂R₉, —NC(NH)NHR₉, —NC(NH)NR₉R₁₀, —NC(NCN)NHR₉, or —NC(NCN)NR₉R₁₀;
R₁ is H;
R₂ is —H, —OH, halogen, —CN, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NHOR₉, —NR₉R₁₀, —C(O)R₉, —OC(O)R₉, —OC(O)OR₉, —OC(O)NHR₉, —OC(O)NR₉R₁₀, —C(O)NR₉R₁₀, —C(O)OR₉, —SR₉, —SOR₉, —S(O)₂R₉, —NHC(O)R₉, —NHS(O)₂R₉, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₁ and R₂ together with the carbon atom to which each is attached, join to form a 3- to 9-membered carbocyclic or heterocyclic ring;
R₃, R₄, R₅, R₆, R₇, and R₈ are, independently, —H, —OH, halogen, —CN, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NHOR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀ or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;
each R₉ and R₁₀ is, independently, —H, —C₁-C₈ alkyl, —C₃-C₁₂ cycloalkyl, —C₆-C₁₂ aryl, —C₇-C₁₄ arylalkyl; and
m is an integer ranging from 1 to 3;
including isomers, prodrugs and pharmaceutically acceptable salts thereof.

4. A compound of claim 1, wherein:
A₁ is —O—, —S—, —SO—, —SO₂—, —CR₇R₈, —NH—, —NR₉—, —NOR₉—, —NC(O)R₉, —NS(O)₂R₉;
A₂ is —O—, —S—, —SO—, —SO₂—, —CR₇R₈, —CR₇R₈-A₃-, -A₃-CR₇R₈—, —NH—, —NR₉—, —NOR₉—, —NC(O)R₉, —NS(O)₂R₉;
A₃ is —O—, —S—, —SO—, —SO₂—, —CR₇R₈, —NH—, —NR₉—, —NOR₉—, —NC(O)R₉, —NS(O)₂R₉;
R₁ is H;
R₂ is —H, —OH, halogen, —CN, —C₁-C₄ alkyl, —C₂-C₆ alkenyl, —C₂-C₆ alkynyl, —C₃-C₅ cycloalkyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)NR₉R₁₀, —C(O)OR₉, —SR₉, —SOR₉, —S(O)₂R₉, —NHC(O)R₉, or —NHS(O)₂R₉, or R₁ and R₂ together with the carbon atom to which each is attached, join to form a 3- to 5-membered carbocyclic or heterocyclic ring;
R₃, R₄, R₅, R₆, R₇, and R₈ are, independently, —H, —OH, halogen, —CN, —SH, —N₃, —C₁-C₄ alkyl, —C₂-C₆ alkenyl, —C₂-C₆ alkynyl, —C₃-C₅ cycloalkyl, phenyl, —C₇-C₁₀ arylalkyl, 3 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 6-membered carbocyclic or heterocyclic ring;
each R₉ and R₁₀ is, independently, —H, —C₁-C₄ alkyl, —C₃-C₆ cycloalkyl, phenyl, —C₇-C₁₀ arylalkyl; and
m is an integer ranging from 1 to 2;
including isomers, prodrugs and pharmaceutically acceptable salts thereof.

5. A compound of claim 1, wherein:
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is −OR₇ or —NR₉R₁₀;
Q₂ is O;
R₁ is H;
R₂ is —H, —C₁-C₄ alkyl, or —C₂-C₆ alkenyl, or R₁ and R₂ together with the carbon atom to which each is attached, join to form a 3- to 5-membered carbocyclic or heterocyclic ring;
R₃, R₄, R₅, R₆, R₇, and R₈ are, independently, —H, —OH, halogen, —CN, —SH, —N₃, —C₁-C₄ alkyl, —C₂-C₆ alkenyl, —C₂-C₆ alkynyl, —C₃-C₅ cycloalkyl, phenyl, benzyl, 3 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 6-membered carbocyclic or heterocyclic ring;
each R₉ and R₁₀ is, independently, —H, —C₁-C₄ alkyl, —C₃-C₆ cycloalkyl, phenyl, benzyl; and
m is an integer ranging from 1 to 2;
including isomers, prodrugs and pharmaceutically acceptable salts thereof.

6. A compound of claim 1, wherein:
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, phenyl, benzyl, 5 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, together with the carbon atom to which each is attached, join to form a 5- to 6-membered carbocyclic or heterocyclic ring;
R₇ and R₈ are, independently, —H, —OH, halogen, —CN, —SH, —N₃, —C₁-C₄ alkyl, —C₂-C₆ alkenyl, —C₂-C₆ alkynyl, —C₃-C₅ cycloalkyl, phenyl, benzyl, 5 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₇ and R₈ together with the carbon atom to which each is attached, join to form a 5- to 6-membered carbocyclic or heterocyclic ring;
each R₉ and R₁₀ is, independently, —H, —C₁-C₄ alkyl, —C₃-C₆ cycloalkyl, phenyl, benzyl; and
m is an integer ranging from 1 to 2;
including isomers, prodrugs and pharmaceutically acceptable salts thereof.

7. A compound of claim 1, wherein:
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆ together with the carbon atom to which each is attached, join to form a 5- to 6-membered carbocyclic or heterocyclic ring;
R₇ and R₈ are, independently, —H, —OH, F, Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₇ and R₈, together with the carbon atom to which each is attached, join to form a 5- to 6-membered carbocyclic or heterocyclic ring;
each R₉ and R₁₀ is, independently, —H, —C₁-C₄ alkyl, —C₃-C₆ cycloalkyl, phenyl, benzyl; and
m is an integer ranging from 1 to 2;
including isomers, prodrugs and pharmaceutically acceptable salts thereof.

8. A compound of claim 1, wherein:
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₃—, —NH—, or —NR₉—;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, F, Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including isomers, prodrugs and pharmaceutically acceptable salts thereof.

9. A compound of claim 1, wherein:
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₉ is, independently, —H or —C₁-C₄ alkyl;
R₁₀ is, independently, —H or —C₁-C₄ alkyl; and
m is an integer ranging from 1 to 2;
including isomers, prodrugs and pharmaceutically acceptable salts thereof.

10. A compound of claim 1, wherein:
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —OR₇;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including isomers, prodrugs and pharmaceutically acceptable salts thereof.

11. A compound of claim 1, wherein:
A₁ is —O—, —S—, —SO₂—, —CR₇R₈, —NH—, or —NR₉—;
A₂ is —O—, —S—, —SO₂—, —CR₇R₃, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —NR₉R₁₀;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including isomers, prodrugs and pharmaceutically acceptable salts thereof.

12. A compound of claim 1, wherein:
A₁ is —O—, —S—, —CR₇R₈, or —NR₉—;
A₂ is —CR₇R₈, —CR₇R₈-A₃-, or -A₃-CR₇R₈—;
A₃ is —CR₇R₈;
Q₁ is —NR₉R₁₀;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl;
m is an integer ranging from 1 to 2;
including isomers, prodrugs and pharmaceutically acceptable salts thereof.

13. A compound of claim 1, wherein:
A₁ is —CR₇R₈;
A₂ is —O—, —S—, —NR₉—, —CR₇R₈, —CR₇R₈-A₃-, or -A₃-CR₇R₈—;
A₃ is —CR₇R₈;
Q₁ is —NR₉R₁₀;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR_{B}, —S(O)₂R₉, —NHC(O)R₉, or —NHS(O)₂R₉;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl;
m is an integer ranging from 1 to 2;
including isomers, prodrugs and pharmaceutically acceptable salts thereof.

14. A compound of claim 1, wherein:
A₁ is —CR₇R₈;
A₂ is —CR₇R₈, —CR₇R₈-A₃-, or -A₃-CR₇R₈—;
A₃ is —O—, —S—, —NR₉—, or —CR₇R₈;
Q₁ is —NR₉R₁₀;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl;
m is an integer ranging from 1 to 2;
including isomers, prodrugs and pharmaceutically acceptable salts thereof.

15. A compound of claim 1, wherein:
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including isomers, prodrugs and pharmaceutically acceptable salts thereof.

16. A compound of claim 1, wherein:
A₁ is —O—, —S—, —CR₇R₈, or —NR₉—;
A₂ is —O—, —S—, —CR₇R₈, —CR₇R₈-A₃-, -A₃-CR₇R₈—, or —NR₉—;
A₃ is —O—, —S—, CR₇R₈, or —NR₉—;
Q₁ is —OR₇ or —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including isomers, prodrugs and pharmaceutically acceptable salts thereof.

17. A compound of claim 1, wherein:
A₁ is —O—, —S—, —CR₇R₈, or —NR₉—;
A₂ is —O—, —S—, —CR₇R₈, —CR₇R₈-A₃-, -A₃-CR₇R₈—, or —NR₉—;
A₃ is —O—, —S—, CR₇R₈, or —NR₉—;
Q₁ is —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, F, Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including isomers, prodrugs and pharmaceutically acceptable salts thereof.

18. A compound of claim 1, wherein:
A₁ is —O—, —S—, —CR₇R₈, or —NR₉—;
A₂ is —CR₇R₈, —CR₇R₈-A₃-, or -A₃-CR₇R₈—;
A₃ is —CR₇R₈;
Q₁ is —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl;
m is an integer ranging from 1 to 2;
including isomers, prodrugs and pharmaceutically acceptable salts thereof.

19. A compound of claim 1, wherein:
A₁ is —CR₇R₈;
A₂ is —O—, —S—, —NR₉—, —CR₇R₈, —CR₇R₈-A₃-, or -A₃-CR₇R₈—;
A₃ is —CR₇R₈;
Q₁ is —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including isomers, prodrugs and pharmaceutically acceptable salts thereof.

20. A compound of claim 1, wherein:
A₁ is —CR₇R₈;
A₂ is —CR₇R₈, —CR₇R₈-A₃-, or -A₃-CR₇R₈—;
A₃ is —O—, —S—, —NR₉—, or —CR₇R₈;
Q₁ is —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, F, Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including isomers, prodrugs and pharmaceutically acceptable salts thereof.

21. 2-[(3R)-3-fluoropyrrolidin-1-yl]acetamide or a pharmaceutically acceptable salt thereof:
3-[(3R)-3-fluoropyrrolidin-1-yl]propanamide or a pharmaceutically acceptable salt thereof;
2,2-difluoro-3-(piperidin-1-yl)propanamide or a pharmaceutically acceptable salt thereof;
2-[(3S)-3-fluoropyrrolidin-1-yl]acetamide,
2-[(3R)-3-hydroxypyrrolidin-1-yl]acetamide,
2-[(3S)-3-hydroxypyrrolidin-1-yl]acetamide,
2-(3,3-difluoropyrrolidin-1-yl)acetamide,
2-[(3R)-3-aminopyrrolidin-1-yl]acetamide,
2-[(3S)-3-aminopyrrolidin-1-yl]acetamide,
2-(1,3-thiazolidin-3-yl)acetamide,
2-[(3R)-3-(dimethylamino)pyrrolidin-1-yl]acetamide,
2-[(3S)-3-(dimethylamino)pyrrolidin-1-yl]acetamide,
3-[(3R)-3-(dimethylamino)pyrrolidin-1-yl]propanamide,
3-[(3S)-3-(dimethylamino)pyrrolidin-1-yl]propanamide,
2-[(3R)-3-(monomethylamino)pyrrolidin-1-yl]acetamide,
2-[(3S)-3-(monomethylamino)pyrrolidin-1-yl]acetamide,
2-[(3S)-3-cyanopyrrolidin-1-yl]acetamide,
2-[(3R)-3-cyanopyrrolidin-1-yl]acetamide,
2-[(3R)-3-methylpyrrolidin-1-yl]acetamide,
2-[(3S)-3-methylpyrrolidin-1-yl]acetamide,
2-[(3S)-3-azidopyrrolidin-1-yl]acetamide,
2-[(3R)-3-azidopyrrolidin-1-yl]acetamide,
2-[(3R)-3-carboxypyrrolidin-1-yl]acetamide,
2-[(3S)-3-carboxypyrrolidin-1-yl]acetamide,
2-[(3S)-3-aminomethylpyrrolidin-1-yl]acetamide,
2-[(3R)-3-chloropyrrolidin-1-yl]acetamide,
2-[(3S)-3-chloropyrrolidin-1-yl]acetamide,
2-[(3R)-3-carboxamidepyrrolidin-1-yl]acetamide,
2-[(3S)-3-carboxamidepyrrolidin-1-yl]acetamide,
2-[(3R)-3-aminomethypyrrolidin-1-yl]acetamide,
3-[(3S)-3-fluoropyrrolidin-1-yl]propanamide,
2-(2-oxo-1,3-thiazolidin-3-yl)acetamide,
2-[(3R)-3-hydroxymethylpyrrolidin-1-yl]acetamide,
2-[(3S)-3-hydroxymethylpyrrolidin-1-yl]acetamide,
2-(1,2-oxazolidin-2-yl)acetamide,
or a pharmaceutically acceptable salt thereof;
3-(1,2-oxazinan-2-yl)propanamide,
3-(morpholin-4-yl)propanamide,
3-(4,4-difluoropiperidin-1-yl)propanamide,
3-(4-fluoropiperidin-1-yl)propanamide,
3-(4-hydroxypiperidin-1-yl)propanamide,
3-(4-aminopiperidin-1-yl)propanamide,
3-[(S)-3-fluoropiperidin-1-yl]propanamide,
3-[(R)-3-fluoropiperidin-1-yl]propanamide,
3-[(S)-3-hydroxypiperidin-1-yl]propanamide,
3-[(R)-3-hydroxypiperidin-1-yl]propanamide,
3-[(S)-3-aminopiperidin-1-yl]propanamide,
3-[(R)-3-aminopiperidin-1-yl]propanamide,
3-(3,3-difluoropiperidin-1-yl)propanamide
3-(4,4-dimethylpiperidin-1-yl)propanamide,
3-(4-hydroxymethylpiperidin-1-yl)propanamide,
2-(1,2-oxazinan-2-yl)acetamide,
2-(morpholin-4-yl)acetamide,
2-(4,4-difluoropiperidin-1-yl)acetamide,
2-(4-fluoropiperidin-1-yl)acetamide,
2-(4-hydroxypiperidin-1-yl)acetamide,
2-(4-aminopiperidin-1-yl)acetamide,
2-[(S)-3-fluoropiperidin-1-yl]acetamide,
2-[(R)-3-fluoropiperidin-1-yl]acetamide,
2-[(S)-3-hydroxypiperidin-1-yl]acetamide,
2-[(R)-3-hydroxypiperidin-1-yl]acetamide,
2-[(S)-3-aminopiperidin-1-yl]acetamide,
2-[(R)-3-aminopiperidin-1-yl]acetamide,
2-(3,3-difluoropiperidin-1-yl)acetamide,
2-(4,4-dimethylpiperidin-1-yl)acetamide,
2-(4-hydroxymethylpiperidin-1-yl)acetamide,
or a pharmaceutically acceptable salt thereof;
N-(4-methylphenyl)-3-{tetrahydrocyclopenta[b]pyrrol-1-yl}propanamide,
N-(4-methylphenyl)-3-(octahydro-1H-indol-1-yl)propanamide,
N-(4-methylphenyl)-2-{octahydrocyclopenta[b]pyrrol-1-yl}acetamide,
N-(4-methylphenyl)-2-(octahydro-1H-indol-1-yl)acetamide,
N-(4-methylphenyl)-(octahydro-1H-indol-1-yl)carboxamide,
N-(4-methylphenyl)-{tetrahydrocyclopenta[b]pyrrol-1-yl}carboxamide,
(R)-2-ethyl-2-(pyrrolidin-1-yl)acetamide,
(S)-2-ethyl-2-(pyrrolidin-1-yl)acetamide,
(R)-2-ethyl-2-(piperidin-1-yl)acetamide,
(S)-2-ethyl-2-(piperidin-1-yl)acetamide,
(R)-2-(4-methylphenyl)-2-(piperidin-1-yl)acetamide,
(S)-2-(4-methylphenyl)-2-(piperidin-1-yl)acetamide,
or a pharmaceutically acceptable salt thereof; or
3-[(3R)-3-(dimethylamino)pyrrolidin-1-yl]propanamide, or a pharmaceutically acceptable salt thereof.

22.-27. (canceled)

28. A composition comprising a pharmaceutically acceptable carrier or vehicle and an effective amount of a compound of claim 1.

29. A method for treating pain in a patient, the method comprising administering to a patient in need thereof an effective amount of a compound of Formula (Ia) of claim 1

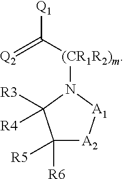

30. The method of claim 29, where for the compound of formula (Ia)
A₁ is —O—, —S—, —SO—, —SO₂—, —CR₇R₈, —NH—, —NR₉—, —NC(O)R₉, —NSR₉, —NSOR₉, —NS(O)₂R₉, —NC(S)R₉, —NC(S)NHR₉, —NC(S)NR₉R₁₀, —NC(NH)NHR₉, —NC(NH)NR₉R₁₀, —NC(NCN)NHR₉, or —NC(NCN)NR₉R₁₀;
A₂ is —O—, —S—, —SO—, —SO₂—, —CR₇R₈, —CR₇R₈-A₃-, -A₃-CR₇R₈—, —NH—, —NR₉—, —NC(O)R₉, —NSR₉, —NSOR₉, —NS(O)₂R₉, —NC(S)R₉, —NC(S)NHR₉, —NC(S)NR₉R₁₀, —NC(NH)NHR₉, —NC(NH)NR₉R₁₀, —NC(NCN)NHR₉, or —NC(NCN)NR₉R₁₀;
A₃ is —O—, —S—, —SO—, —SO₂—, —CR₇R₈, —NH—, —NR₉—, —NC(O)R₉, —NSR₉, —NSOR₉, —NS(O)₂R₉, —NC(S)R₉, —NC(S)NHR₉, —NC(S)NR₉R₁₀, —NC(NH)NHR₉, —NC(NH)NR₉R₁₀, —NC(NCN)NHR₉, or —NC(NCN)NR₉R₁₀;
Q₁ is —NR₉R₁₀;
Q₂ is O;
R₁ is —H;
R₂ is —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —or NR₉C(NCN)NR₉R₁₀, or R₁ and R₂ together with the carbon atom to which each is attached, join to form a 3- to 9-membered carbocyclic or heterocyclic ring;
R₃, R₄, R₅, R₆, R₇, and R₈ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —O(CH₂)₆OR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O″, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;
R₉ and R₁₀ are, independently, —H, CH₃, CH₂CH₃, or CH(CH₃)₂, phenyl, or benzyl; and
each n is, independently, an integer ranging from 1 to 2;
including enantiomers, diastereomers, isomers, prodrugs and pharmaceutically acceptable salts thereof;
A₁ is —O—, —S—, —SO—, —SO₂—, —CR₇R₈, —NH—, —NR₉—, —NC(O)R₉, —NS(O)₂R₉, —NC(NH)NHR₉, —NC(NH)NR₉R₁₀, —NC(NCN)NHR₉, or —NC(NCN)NR₉R₁₀;
A₂ is —O—, —S—, —SO—, —SO₂—, —CR₇R₈-A₃, -A₃-CR₇R₈—, —NH—, —NR₉—, —NC(O)R₉, —NS(O)₂R₉, —NC(S)R₉, —NC(NH)NHR₉, —NC(NH)NR₉R₁₀, —NC(NCN)NHR₉, or —NC(NCN)NR₉R₁₀;
A₃ is —O—, —S—, —SO—, —SO₂—, —CR₇R₈, —NH—, —NR₉—, —NC(O)R₉, —NS(O)₂R₉, —NC(NH)NHR₉, —NC(NH)NR₉R₁₀, —NC(NCN)NHR₉, or —NC(NCN)NR₉R₁₀;
R₁ is —H;
R₂ is —H, —OH, halogen, —CN, —C₁-C₈ alkyl, —C₂-C₈alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)NR₉R₁₀, —C(O)OR₉, —SR₉, —SOR₉, —S(O)₂R₉, —NHC(O)R₉, —NHS(O)₂R₉, —NHC(NH)NHR₉, —NR₉C(NH)NHR₉, —NHC(NH)NR₉R₁₀, —NR₉C(NH)NR₉R₁₀, —NHC(NCN)NHR₉ or —NR₉C(NCN)NR₉R₁₀, or R₁ and R₂ together with the carbon atom to which each is attached, join to form a 3- to 9-membered carbocyclic or heterocyclic ring;
R₃, R₄, R₅, R₆, R₇, and R₈ are, independently, —H, —OH, halogen, —CN, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;
each R₉ and R₁₀ is, independently, —H, —C₁-C₈ alkyl, —C₃-C₁₂ cycloalkyl, —C₆-C₁₂ aryl, or —C₇-C₁₄ arylalkyl; and
m is an integer ranging from 1 to 3;
including enantiomers, diastereomers, isomers, prodrugs and pharmaceutically acceptable salts thereof;
A₁ is —O—, —S—, —SO—, —SO₂—, —CR₇R₈, —NH—, —NR₉—, —NC(O)R₉, or —NS(O)₂R₉;
A₂ is —O—, —S—, —SO—, —SO₂—, —CR₇R₈, —CR₇R₈-A₃-, -A₃-CR₇R₈—, —NH—, —NR₉—, —NC(O)R₉, or —NS(O)₂R₉;
A₃ is —O—, —S—, —SO—, —SO₂—, —CR₇R₈, —NH—, —NR₉—, —NC(O)R₉, or —NS(O)₂R₉;
R₁ is H;
R₂ is —H, —OH, halogen, —CN, —C₁-C₄ alkyl, —C₂-C₆ alkenyl, —C₂-C₆ alkynyl, —C₃-C₅ cycloalkyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)NR₉R₁₀, —C(O)OR₉, —SR₉, —SOR₉, —S(O)₂R₉, —NHC(O)R₉, —NHS(O)₂R₉, or R₁ and R₂ together with the carbon atom to which each is attached, join to form a 3- to 5-membered carbocyclic or heterocyclic ring;
R₃, R₄, R₅, R₆, R₇, and R₈ are, independently, —H, —OH, halogen, —CN, —SH, —N₃, —C₁-C₄ alkyl, —C₁-C₆ alkenyl, —C₂-C₆ alkynyl, —C₃-C₅ cycloalkyl, -phenyl, —C₇-C₁₀ arylalkyl, 3 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 6-membered carbocyclic or heterocyclic ring;
each R₉ and R₁₀ is, independently, —H, —C₁-C₄ alkyl, —C₃-C₆ cycloalkyl, phenyl, or —C₇-C₁₀ arylalkyl; and
m is an integer ranging from 1 to 2;
including enantiomers, diastereomers, isomers, prodrugs and pharmaceutically acceptable salts thereof;
A₁ is —O—, —S—, —SO₂—, —CR₇R₈, —NH—, or —NR₉—;
A₂ is —O—, —S—, —SO₂—, —CR₇R₈—CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H, —C₁-C₄ alkyl, —C₂-C₆ alkenyl, or R₁ and R₂ together with the carbon atom to which each is attached, join to form a 3- to 5-membered carbocyclic or heterocyclic ring;
R₃, R₄, R₅, R₆, R₇, and R₈ are, independently —H, —OH, halogen, —CN, —SH, —N₃, —C₁-C₄ alkyl, —C₂-C₆ alkenyl, —C₂-C₆ alkynyl, —C₃-C₅ cycloalkyl, phenyl, benzyl, 3 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 6-membered carbocyclic or heterocyclic ring;
each R₉ and R₁₀ is, independently, —H, —C₁-C₄ alkyl, —C₃-C₆ cycloalkyl, phenyl, or benzyl; and
m is an integer ranging from 1 to 2;
including enantiomers, diastereomers, isomers, prodrugs and pharmaceutically acceptable salts thereof;
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, -phenyl, -benzyl, 5 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, together with the carbon atom to which each is attached, join to form a 5- to 6-membered carbocyclic or heterocyclic ring;
R₇ and R₈ are, independently, —H, —OH, halogen, —CN, —SH, —N₃, —C₁-C₄ alkyl, —C₂-C₆ alkenyl, —C₂-C₆ alkynyl, —C₃-C₅ cycloalkyl, -phenyl, -benzyl, 5 to 6-membered heterocycle, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₇ and R₈ together with the carbon atom to which each is attached, join to form a 5- to 6-membered carbocyclic or heterocyclic ring;
each R₉ and R₁₀ is, independently, —H, —C₁-C₄ cycloalkyl, phenyl, benzyl; and
m is an integer ranging from 1 to 2;
including enantiomers, diastereomers, isomers, prodrugs and pharmaceutically acceptable salts thereof;
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H or —C₁-C₄ alkyl;
R₃, R₄, R₅, and R₆ are, independently, —H, —OH, F, Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆ together with the carbon atom to which each is attached, join to form a 5- to 6-membered carbocyclic or heterocyclic ring;
R₇ and R₈ are, independently, —H, —OH, F, Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₇ and R₈, together with the carbon atom to which each is attached, join to form a 5- to 6-membered carbocyclic or heterocyclic ring;
each R₉ and R₁₀ is, independently, —H, —C₁-C₄ alkyl, —C₃-C₆ cycloalkyl, -phenyl, or -benzyl; and
m is an integer ranging from 1 to 2;
including enantiomers, diastereomers, isomers, prodrugs and pharmaceutically acceptable salts thereof;
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H or —C₁-C₄ alkyl;
R₃, R₄, R₅, and R₆ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHS(O)₂R₉, or R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof;
A₁ is —O—, —S—, —SO₂—, —CR₇R₈, —NH—, or —NR₉—;
A₂ is —O—, —S—, —SO₂—, —CR₇R₈—CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H or —C₁-C₄ alkyl;
R₃, R₄, R₅, and R₆ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof;
A₁ is —O—, —S—, —SO₂—, —CR₇R₈, —NH—, or —NR₉—;
A₂ is —O—, —S—, —SO₂—, —CR₇R₈, —CR₇R₈A₃-, -A₃-CR₇R₈—, —NH—, or —NR₉—;
A₃ is —O—, —S—, —SO₂—, —CR₇R₈, —NH—, or —NR₉—;
Q₁ is —OR₇;
Q₂ is O;
R₁ is H;
R₂ is —H or —C₁-C₄ alkyl;
R₃, R₄, R₅, and R₆ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H, —C₁-C₄ alkyl; and
m is an integer ranging from 1 to 2;
including enantiomers, diastereomers, isomers, prodrugs and pharmaceutically acceptable salts thereof;
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉—;
A₃ is —O—, —S—, —SO₂—, —CR₇R₈, —NH—, or —NR₉—;
Q₁ is —NR₉R₁₀;
Q₂ is O;
R₁ is H.
R₂ is —H or —C₁-C₄ alkyl;
R₃, R₄, R₅, and R₆ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof;
A₁ is —O—, —S—, —CR₇R₈ or —NR₉—;
A₂ is —CR₇R₈, —CR₇R₈-A₃-, or -A₃-CR₇R₈—;
A₃ is —CR₇R₈;
Q₁ is —NR₉R₁₀;
Q₂ is O;
R₁ is H;
R₂ is —H or —C₁-C₄ alkyl;
R₃, R₄, R₅, and R₆ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof;
A₁ is —CR₇R₈;
A₂ is —O—, —S—, —NR₉—, —CR₇R₈, —CR₇R₈-A₃-, or -A₃-CR₇R₈—;
A₃ is —CR₇R₈;
Q₁ is —NR₉R₁₀;
Q₂ is O;
R₁ is H;
R₂ is —H or —C₁-C₄ alkyl;
R₃, R₄, R₅, and R₆ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof;
A₁ is —CR₇R₈;
A₂ is —CR₇R₈, —CR₇R₈-A₃-, or -A₃-CR₇R₈—;
A₃ is —O—, —S—, —NR₉—, or —CR₇R₈;
Q₁ is —NR₉R₁₀;
Q₂ is O;
R₁ is H;
R₂ is —H or —C₁-C₄ alkyl;
R₃, R₄, R₅, and R₆ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof;
A₁ is —O—, —S—, —SO₂—, —CR₇R₈, —NH—, or —NR₉—;
A₂ is —O—, —S—, —SO₂—, —CR₇R₈, —CR₇R₈-A₃-, -A₃-CR₇R₈—, —NH—, or —NR₉;
A₃ is —O—, —S—, —SO₂—, —CR₇R₈, —NH—, or —NR₉—;
Q₁ is —OR₇ or —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉,
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof;
A₁ is —O—, —S—, —CR₇R₈, or —NR₉—;
A₂ is —O—, —S—, —CR₇R₈, —CR₇R₈-A₃-, -A₇-CR₇R₈—, or —NR₉—;
A₃ is —O—, —S—, CR₇R₈, or —NR₉—;
Q₁ is —OR₇ or —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof;
A₁ is —O—, —S—, —CR₇R₈, or —NR₉—;
A₂ is —O—, —S—, —CR₇R₈, —CR₇R₈-A₃-, -A₃-CR₇R₈—, or —NR₉—;
A₂ is —O—, —S—, CR₇R₈, or —NR₉—;
Q₁ is —NR₉R₁₀;
Q₂ is O;
R₁ is H;
R₂ is —H or —C₁-C₄ alkyl;
R₁ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, —F, —Cl, —CN, —C₁-C₄ alkyl, —C₂-C₆ alkenyl, phenyl, benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl;
R₁₀ is, independently, —H, —C₁-C₄ alkyl; and
m is an integer ranging from 1 to 2;
including enantiomers, diastereomers, isomers, prodrugs and pharmaceutically acceptable salts thereof;
A₁ is —O—, —S—, —CR₇R₈, or —NR₉—;
A₂ is —CR₇R₈, —CR₇R₈-A₃-, or -A₃-CR₇R₈—;
A₃ is —CR₇R₈;
Q₁ is —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof;
A₁ is —CR₇R₈;
A₂ is —O—, —S—, —NR₉—, —CR₇R₈, —CR₇R₈-A₃-, or -A₃-CR₇R₈—;
A₃ is —CR₇R₈;
Q₁ is —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof; or
A₁ is —CR₇R₈;
A₂ is —CR₇R₈, —CR₇R₈-A₃-, or -A₃-CR₇R₈—;
A₃ is —O—, —S—, —NR₉—, or —CR₇R₈—;
Q₁ is —NR₉R₁₀;
Q₂ is O;
R₁ is H;
R₂ is —H or —C₁-C₄ alkyl;
R₃ and R₅, or R₃ and R₆, or R₄ and R₅, or R₄ and R₆, together with the carbon atom to which each is attached, join to form a 5- to 6-membered carbocyclic or heterocyclic ring;
R₇ and R₈ are, independently, —H, —OH, —F, —Cl, —CN, —C₁-C₄ alkyl, —C₂-C₆ alkenyl, -phenyl, -benzyl, —OR₉, —NHR₉, —NR₉R₁₀, —C(O)R₉, —OC(O)R₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, or —NHS(O)₂R₉;
each R₉ and R₁₀ is, independently, —H or —C₁-C₄ alkyl; and
m is an integer ranging from 1 to 2;
including enantiomers, diastereomers, isomers, prodrugs and pharmaceutically acceptable salts thereof.

31.-48. (canceled)

49. The method of claim 29, where the compound of Formula (Ia) is 2-[(3R)-3-fluoropyrrolidin-1-yl]acetamide or a pharmaceutically acceptable salt thereof;
3-[(3R)-3-fluoropyrrolidin-1-yl]propanamide or a pharmaceutically acceptable salt thereof;
2,2-difluoro-3-(piperidin-1-yl)propanamide or a pharmaceutically acceptable salt thereof;
one or more of
2-[(3S)-3-fluoropyrrolidin-1-yl]acetamide,
2-[(3R)-3-hydroxypyrrolidin-1-yl]acetamide,
2-[(3S)-3-hydroxypyrrolidin-1-yl]acetamide,
2-(3,3-difluoropyrrolidin-1-yl)acetamide,
2-[(3R)-3-aminopyrrolidin-1-yl]acetamide,
2-[(3S)-3-aminopyrrolidin-1-yl]acetamide,
2-(1,3-thiazolidin-3-yl)acetamide,
2-[(3R)-3-(dimethylamino)pyrrolidin-1-yl]acetamide
2-[(3S)-3-(dimethylamino)pyrrolidin-1-yl]acetamide,
3-[(3R)-3-(dimethylamino)pyrrolidin-1-yl]propanamide,
3-[(3S)-3-(dimethylamino)pyrrolidin-1-yl]propanamide,
2-[(3R)-3-(monomethylamino)pyrrolidin-1-yl]acetamide,
2-[(3S)-3-(monomethylamino)pyrrolidin-1-yl]acetamide,
2-[(3S)-3-cyanopyrrolidin-1-yl]acetamide,
2-[(3R)-3-cyanopyrrolidin-1-yl]acetamide,
2-[(3R)-3-methylpyrrolidin-1-yl]acetamide,
2-[(3S)-3-methylpyrrolidin-1-yl]acetamide,
2-[(3S)-3-azidopyrrolidin-1-yl]acetamide,
2-[(3R)-3-azidopyrrolidin-1-yl]acetamide,
2-[(3R)-3-carboxypyrrolidin-1-yl]acetamide,
2-[(3S)-3-carboxypyrrolidin-1-yl]acetamide,
2-[(3S)-3-aminomethylpyrrolidin-1-yl]acetamide,
2-[(3R)-3-chloropyrrolidin-1-yl]acetamide,
2-[(3S)-3-chloropyrrolidin-1-yl]acetamide,
2-[(3R)-3-carboxamidepyrrolidin-1-yl]acetamide,
2-[(3S)-3-carboxamidepyrrolidin-1-yl]acetamide,
2-[(3R)-3-aminomethypyrrolidin-1-yl]acetamide,
3-[(3S)-3-fluoropyrrolidin-1-yl]propanamide,
2-(2-oxo-1,3-thiazolidin-3-yl)acetamide,
2-[(3R)-3-hydroxymethylpyrrolidin-1-yl]acetamide,
2-[(3S)-3-hydroxymethylpyrrolidin-1-yl]acetamide,
2-(1,2-oxazolidin-2-yl)acetamide,
or a pharmaceutically acceptable salt thereof;
one or more of
3-(1,2-oxazinan-2-yl)propanamide,
3-(morpholin-4-yl)propanamide,
3-(4,4-difluoropiperidin-1-yl)propanamide,
3-(4-fluoropiperidin-1-yl)propanamide,
3-(4-hydroxypiperidin-1-yl)propanamide,
3-(4-aminopiperidin-1-yl)propanamide,
3-[(S)-3-fluoropiperidin-1-yl]propanamide,
3-[(R)-3-fluoropiperidin-1-yl]propanamide,
3-[(S)-3-hydroxypiperidin-1-yl]propanamide,
3-[(R)-3-hydroxypiperidin-1-yl]propanamide,
3-[(S)-3-aminopiperidin-1-yl]propanamide,
3-[(R)-3-aminopiperidin-1-yl]propanamide,
3-(3,3-difluoropiperidin-1-yl)propanamide,
3-(4,4-dimethylpiperidin-1-yl)propanamide,
3-(4-hydroxymethylpiperidin-1-yl)propanamide,
2-(1,2-oxazinan-2-yl)acetamide,
2-(morpholin-4-yl)acetamide,
2-(4,4-difluoropiperidin-1-yl)acetamide,
2-(4-fluoropiperidin-1-yl)acetamide,
2-(4-hydroxypiperidin-1-yl)acetamide,
2-(4-aminopiperidin-1-yl)acetamide,
2-[(S)-3-fluoropiperidin-1-yl]acetamide,
2-[(R)-3-fluoropiperidin-1-yl]acetamide,
2-[(S)-3-hydroxypiperidin-1-yl]acetamide,
2-[(R)-3-hydroxypiperidin-1-yl]acetamide,
2-[(S)-3-aminopiperidin-1-yl]acetamide,
2-[(R)-3-aminopiperidin-1-yl]acetamide,
2-(3,3-difluoropiperidin-1-yl)acetamide,
2-(4,4-dimethylpiperidin-1-yl)acetamide,
2-(4-hydroxymethylpiperidin-1-yl)acetamide,
or a pharmaceutically acceptable salt thereof;
one or more of
N-(4-methylphenyl)-3-{tetrahydrocyclopenta[b]pyrrol-1-yl}propanamide,
N-(4-methylphenyl)-3-(octahydro-1H-indol-1-yl)propanamide,
N-(4-methylphenyl)-2-{octahydrocyclopenta[b]pyrrol-1-yl}acetamide,
N-(4-methylphenyl)-2-(octahydro-1H-indol-1-yl)acetamide,
N-(4-methylphenyl)-(octahydro-1H-indol-1-1 carboxamide
N-(4-methylphenyl)-{tetrahydrocyclopenta[b]pyrrol-1-yl}carboxamide,
(R)-2-ethyl-2-(pyrrolidin-1-yl)acetamide,
(S)-2-ethyl-2-(pyrrolidin-1-yl)acetamide,
(R)-2-ethyl-2-(piperidin-1-yl)acetamide,
(S)-2-ethyl-2-(piperidin-1-yl)acetamide,
(R)-2-(4-methylphenyl)-2-(piperidin-1-yl)acetamide,
(S)-2-(4-methylphenyl)-2-(piperidin-1-yl)acetamide,
or a pharmaceutically acceptable salt thereof; or
3-[(3R)-3-(dimethylamino)pyrrolidin-1-yl]propanamide, or a pharmaceutically acceptable salt thereof.

50.-55. (canceled)

56. A method for treating pain in a patient, the method comprising administering to a patient in need thereof an effective amount of the compound of formula (Ib) 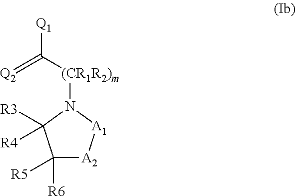
wherein:
A₁ is —CR₇R₈— or —C(O)—;
A₂ is —CR₇R₈—, —CR₇R₈-A₃-, or -A₃-CR₇R₈—;
A₃ is —CR₇R₈—;
Q₁ is —OR₇, —SR₇, or —NR₉R₁₀;
Q₂ is O, S, NH, or NR₉;
R₁ and R₂ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NHOR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₁ and R₂ together with the carbon atom to which each is attached, join to form a 3- to 9-membered carbocyclic or heterocyclic ring;
R₃, R₄, R₅, R₆, R₇, and R₈ are, independently, —H, —OH, halogen, —CN, —NO₂, —SH, —N₃, —C₁-C₈ alkyl, —C₂-C₈ alkenyl, —C₂-C₈ alkynyl, —C₃-C₁₂ cycloalkyl, —C₆-C₁₂ aryl, —C₇-C₁₄ arylalkyl, 3- to 9-membered heterocycle, —OR₉, —NHR₉, —NHOR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)OR₉, —C(O)NR₉R₁₀, —SR₉, —SOR₉, —S(O)₂R₉, —NHC(O)R₉, —NHSR₉, —NHSOR₉, —NHS(O)₂R₉, —OS(O)₂O⁻, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀; —NHC(NCN)NHR₉, —NR₉C(NCN)NR₉R₁₀, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;
each R₉ and R₁₀ is, independently, —H, —C₁-C₈ alkyl, —C₃-C₁₂ cycloalkyl, —C₆-C₁₂ aryl, —C₇-C₁₄ arylalkyl, 3- to 9-membered heterocycle, —C₂-C₈ alkenyl, or —C₂-C₈ alkynyl;
each n is, independently, an integer ranging from 1 to 5;
m is an integer ranging from 0 to 3;
with the proviso that A₁ and A₂ are not —C(OH)₂—;
including enantiomers, diastereomers, isomers, prodrugs and pharmaceutically acceptable salts thereof.

57. The method of claim 56, where for the compound of formula (Ib):
R₁ and R₂ are, independently, —H, —OH, halogen, —CN, —NO₂, —SH, —N₃, —C₁-C₈ alkyl, —C₂-C₆ alkenyl, —C₂-C₆ alkynyl, —C₃-C₆ cycloalkyl, —C₆-C₁₀ aryl, —C₇-C₁₀ arylalkyl, 3 to 7-membered heterocycle, —OR₉, —NHR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)NR₉R₁₀, —C(O)OR₉, —SR₉, —SOR₉, —S(O)₂R₉, —NHC(O)R₉, —NHSR₉, —NHSOR₉, —NHS(O)₂R₉, —OS(O)₂O⁻, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₁ and R₂ together with the carbon atom to which each is attached, join to form a 3- to 7-membered carbocyclic or heterocyclic ring;
R₃, R₄, R₅, R₆, R₇, and R₈ are, independently, —H, —OH, halogen, —CN, —NO₂, —SH, —N₃, —C₁-C₆ alkyl, —C₂-C₆ alkenyl, —C₂-C₆ alkynyl, —C₃-C₁₀ cycloalkyl, —C₆-C₁₀ aryl, —C₇-C₁₀ arylalkyl, 3 to 7-membered heterocycle, —OR₉, —NHR₉, —NR₉R₁₀, —O(CH₂)ₙOR₉, —C(O)R₉, —OC(O)R₉, —C(O)(CH₂)ₙR₉, —OC(O)OR₉, —OC(O)NHR₉, —OC(O)NR₉R₁₀, —C(O)NR₉R₁₀, —C(O)OR₉, —SR₉, —SOR₉, —S(O)₂R₉, —NHC(O)R₉, —NHSR₉, —NHSOR₉, —NHS(O)₂R₉, —OS(O)₂O⁻, —OC(S)R₉, —OC(S)OR₉, —OC(S)NHR₉, —OC(S)NHNR₉R₁₀, —C(S)OR₉, —C(S)NHR₉, —C(S)NHNR₉R₁₀, —NHC(S)R₉, —NR₉C(S)R₉, —NHC(S)NHR₉, —NHC(S)NR₉R₁₀, —NR₉C(S)NHR₉, —NR₉C(S)NR₉R₁₀, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;
each R₉ and R₁₀ is, independently, —H, —C₁-C₈ alkyl, —C₃-C₁₂ cycloalkyl, —C₆-C₁₂ aryl, —C₇-C₁₄ arylalkyl, 3 to 7-membered heterocycle, —C₂-C₈ alkenyl, or —C₂-C₈ alkynyl;
each n is, independently, an integer ranging from 1 to 3; and
m is an integer ranging from 1 to 3;
including enantiomers, diastereomers, isomers, prodrugs and pharmaceutically acceptable salts thereof;
Q₁ is —OR₇ or —NR₉R₁₀;
Q₂ is O or NR₉;
R₁ and R₂ are, independently, —H, —OH, —F, —Cl, —CN, —C₁-C₈ alkyl, —C₂-C₆ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —SR₉, —SOR₉, —S(O)₂R₉, —NHC(O)R₉, —NHS(O)₂R₉, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀ or R₁ and R₂ together with the carbon atom to which each is attached, join to form a 3- to 7-membered carbocyclic or heterocyclic ring;
R₃, R₄, R₅, R₆, R₇, and R₈ are, independently, —H, —OH, —F, —Cl, —CN, —NO₂, —SH, —N₃, —C₁-C₆ alkyl, —C₂-C₆ alkenyl, —C₂-C₆ alkynyl, —OR₉, —NHR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉, —NHC(NH)NHR₉, —NR₉C(NH)NHR₉, —NHC(NH)NR₉R₁₀, —NR₉C(NH)NR₉R₁₀, —NHC(NCN)NHR₉, —NR₉C(NCN)NR₉R₁₀, or R₃ and R₄, or R₃ and R₅, or R₇ and R₆, or R₄ and R₅, or R₄ and R₆, or R₅ and R₆, or R₇ and R₈, together with the carbon atom to which each is attached, join to form a 3- to 9-membered carbocyclic or heterocyclic ring;
each R₉ and R₁₀ is, independently, —H, —C₁-C₆ alkyl, —C₃-C₇ cycloalkyl, —C₆-C₁₀ aryl, —C₇-C₁₀ arylalkyl, 3 to 7-membered heterocycle, —C₂-C₈ alkenyl, or —C₂-C₈ alkynyl; and
m is an integer ranging from 1 to 2;
including enantiomers, diastereomers, isomers, prodrugs and pharmaceutically acceptable salts thereof;
Q₁ is —OR₇ or —NR₉R₁₀;
Q₂ is O;
R₁ is —H or —F;
and R₂ is —H, F, —C₁-C₄ alkyl, —C₂-C₆ alkenyl, —OR₉, or —NR₉R₁₀;
R₃, R₄, R₅, R₆, R₇, and R₈ are, independently, —H, —OH, —F, —Cl, —CN, —C₁-C₄ alkyl, —C₇-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉, or R₃ and R₄, or R₃ and R₅, or R₃ and R₆, or R₄ and R₅, or R₄ and R₆, or R₅ and R₆, or R₇ and R₈, together with the carbon atom to which each is attached, join to form a 3- to 6-membered carbocyclic or heterocyclic ring;
each R₉ and R₁₀ are, independently, —H, —C₁-C₄ alkyl, —C₃-C₆ cycloalkyl, phenyl, benzyl, —C₂-C₄ alkenyl; and
m is an integer ranging from 1 to 2;
including enantiomers, diastereomers, isomers, prodrugs and pharmaceutically acceptable salts thereof;
Q₁ is —NR₉R₁₀;
Q₂ is O;
R₁ is —H;
and R₂ is —H, —C₁-C₄ alkyl, or —C₂-C₄ alkenyl;
R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉, or R₃ and R₅, or R₃ and R₆, or R₄ and R₅, or R₄ and R₆, together with the carbon atom to which each is attached, join to form a 5- to 6-membered carbocyclic or heterocyclic ring;
each R₉ and R₁₀ is, independently, —H, —C₁-C₄ alkyl, —C₃-C₆ cycloalkyl, phenyl, benzyl, or —C₁-C₄ alkenyl; and
m is an integer ranging from 1 to 2;
including enantiomers, diastereomers, isomers, prodrugs and pharmaceutically acceptable salts thereof;
Q₁ is —NR₉R₁₀;
Q₂ is O;
R₁ is —H;
and R₂ is —H or —C₁-C₄ alkyl;
R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉;
each R₉ and R₁₀ is, independently, —H, —C₁-C₄ alkyl, phenyl, benzyl, —C₂-C₄ alkenyl; and
m is an integer ranging from 1 to 2;
including enantiomers, diastereomers, isomers, prodrugs and pharmaceutically acceptable salts thereof;
Q₁ is —NR₉R₁₀;
Q₂ is O;
R₁ is —H;
and R₂ is —H, —C₁-C₄ alkyl, or —C₂-C₄ alkenyl;
R₃ and R₅, or R₁ and R₆, or R₄ and R₅, or R₄ and R₆, together with the carbon atom to which each is attached, join to form a 5- to 6-membered carbocyclic or heterocyclic ring;
each R₉ and R₁₀ is, independently, —H, —C₁-C₄ alkyl, phenyl, benzyl, or —C₂-C₄ alkenyl; and
m is an integer ranging from 1 to 2;
including enantiomers, diastereomers, isomers, prodrugs and pharmaceutically acceptable salts thereof;
A₂ is —CR₇R₈;
Q₁ is —NR₉R₁₀;
Q₂ is O;
R₁ is —H;
R₂ is —H, —C₁-C₄ alkyl, or —C₂-C₄ alkenyl;
R₃ and R₅, or R₃ and R₆, or R₄ and R₅, or R₄ and R₆, together with the carbon atom to which each is attached, join to form a 5- to 6-membered carbocyclic or heterocyclic ring;
each R₉ and R₁₀ is, independently, —H, —C₁-C₄ alkyl, phenyl, benzyl, or —C₂-C₄ alkenyl; and
m is an integer ranging from 1 to 2;
including enantiomers, diastereomers, isomers, prodrugs and pharmaceutically acceptable salts thereof; A₂ is —CR₇R₈-A₃- or -A₃-CR₇R₈—;
Q₁ is —NR₉R₁₀;
Q₂ is O;
R₁ is —H;
R₂ is —H, —C₁-C₄ alkyl, or —C₂-C₄ alkenyl;
R₃ and R₅, or R₃ and R₆, or R₄ and R₅, or R₄ and R₆, together with the carbon atom to which each is attached, join to form a 5- to 6-membered carbocyclic or heterocyclic ring;
each R₉ and R₁₀ is, independently, —H, —C₁-C₄ alkyl, phenyl, benzyl, or —C₂-C₄ alkenyl; and
m is an integer ranging from 1 to 2;
including enantiomers, diastereomers, isomers, prodrugs and pharmaceutically acceptable salts thereof;
A₂ is —CR₇R₈;
Q₁ is —NH₂;
R₁ is —H;
R₂ is —H or —C₁-C₄ alkyl;
R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, or —NHS(O)₂R₉;
each R₉ and R₁₀ is, independently, —H, —C₁-C₄ alkyl, phenyl, benzyl, or —C₂-C₄ alkenyl; and
m is an integer ranging from 1 to 2;
including enantiomers, diastereomers, isomers, prodrugs and pharmaceutically acceptable salts thereof;
A₂ is —CR₇R₈-A₃- or -A₃-CR₇R₈—;
A₃ is —CR₇R₈;
Q₁ is —NH₂;
Q₂ is O;
R₁ is —H;
R₂ is —H or —C₁-C₄ alkyl;
R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉;
each R₉ and R₁₀ is, independently, —H, —C₁-C₄ alkyl, phenyl, benzyl, or —C₂-C₄ alkenyl; and
m is an integer ranging from 1 to 2;
including enantiomers, diastereomers, isomers, prodrugs and pharmaceutically acceptable salts thereof;
A₂ is —CR₇R₈;
Q₁ is —NH₂;
Q₂ is O;
R₁ is —H;
R₂ is —H, —C₁-C₄ alkyl, —C₂-C₄ alkenyl;
R₃ and R₅, or R₃ and R₆, or R₄ and R₅, or R₄ and R₆, together with the carbon atom to which each is attached, join to form a 5- to 6-membered carbocyclic or heterocyclic ring; and
m is an integer ranging from 1 to 2;
including enantiomers, diastereomers, isomers, prodrugs and pharmaceutically acceptable salts thereof;
A₂ is —CR₇R₈-A₃- or -A₃-CR₇R₈;
A₃ is —CR₇R₈;
Q₁ is —NH₂;
Q₂ is O;
R₁ is —H;
R₂ is —H, —C₁-C₄ alkyl, or —C₂-C₄ alkenyl;
R₃ and R₅, or R₃ and R₆, or R₄ and R₅, or R₄ and R₆, together with the carbon atom to which each is attached, join to form a 5- to 6-membered carbocyclic or heterocyclic ring; and
m is an integer ranging from 1 to 2;
including enantiomers, diastereomers, isomers, prodrugs and pharmaceutically acceptable salts thereof;
A₂ is —CR₇R₈;
Q₁ is —NH₂;
Q₂ is O;
R₁ is —H;
R₂ is —H or —C₁-C₄ alkyl;
R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, or —NHS(O)₂R₉;
each R₉ and R₁₀ is, independently, —H, —C₁-C₄ alkyl, -phenyl, -benzyl, or —C₂-C₄ alkenyl; and
m is an integer ranging from 1 to 2;
including enantiomers, diastereomers, isomers, prodrugs and pharmaceutically acceptable salts thereof; or
A₂ is —CR₇R₈-A₃- or -A₃-CR₇R₈—;
A₃ is —CR₇R₈;
Q₁ is —NH₂;
Q₂ is O;
R₁ is —H;
R₂ is —H or —C₁-C₄ alkyl;
R₃, R₄, R₅, R₆, R₇, and R₈ are, independently, —H, —OH, —F, —Cl, —CN, —C₁-C₄ alkyl, —C₂-C₄ alkenyl, —OR₉, —NR₉R₁₀, —C(O)R₉, —OC(O)R₉, —OC(O)OR₉, —OC(O)NR₉R₁₀, —C(O)NR₉R₁₀, —C(O)OR₉, —S(O)₂R₉, —NHC(O)R₉, —NHS(O)₂R₉;
each R₉ and R₁₀ is, independently, —H, —C₁-C₄alkyl, -phenyl, -benzyl, or —C₂-C₄ alkenyl; and
m is an integer ranging from 1 to 2;
including enantiomers, diastereomers, isomers, prodrugs and pharmaceutically acceptable salts thereof.

58.-70. (canceled)

71. The method of claim 56, where the compound is 2-(pyrrolidin-1-yl)acetamide or a pharmaceutically acceptable salt thereof;
3-(piperidin-1-yl)propanamide or a pharmaceutically acceptable salt thereof or one or more of:
2-(pyrrolidin-1-yl)acetamide,
3-(pyrrolidin-1-yl)propanamide,
2-(piperidin-1-yl)acetamide,
3-(piperidin-1-yl)propanamide,
3-(octahydro-1H-indo-1-yl)propanamide,
3-{tetrahydrocyclopenta[b]pyrrol-1-yl}propanamide,
2-(octahydro-1H-indo-1-yl)acetamide,
2-{tetrahydrocyclopenta[b]pyrrol-1-yl}acetamide,
2-{tetrahydrocyclopenta[c]pyrrol-1-yl}acetamide,
3-{tetrahydrocyclopenta[c]pyrrol-1-yl}propanamide,
3-(octahydro-1H-isoindol-2-yl)propanamide,
2-(octahydro-1H-isoindol-2-yl)acetamide,
or enantiomers, diastereomers, isomers, prodrugs and pharmaceutically acceptable salts thereof.

72.-73. (canceled)

74. 2-cyclopentylacetamide or a pharmaceutically acceptable salt thereof.

75. A method for treating pain in a patient, the method comprising administering to a patient in need thereof an effective amount of the compound or salt of claim 74.
